(19) United States
(12) Patent Application Publication
(10) Pub. No.: US 2011/0041725 A1
(43) Pub. Date: Feb. 24, 2011

(54) ANTICOAGULANTS AS ANTIFOULING AGENTS

(76) Inventors: Daniel Rittschof, Morehead City, NC (US); Gary H. Dickinson, Beaufort, NC (US); Beatriz Orihuela de Diaz, Morehead City, NC (US); Eric R. Holm, Mclean, VA (US)

Correspondence Address: JENKINS, WILSON, TAYLOR & HUNT, P. A., 3100 Tower Blvd., Suite 1200, DURHAM, NC, 27707, US

(21) Appl. No.: 12/665,505

(22) PCT Filed: Jun. 13, 2008
(86) PCT No.: PCT/US08/07433

§ 371 Date: Sep. 27, 2010

(30) Foreign Application Priority Data
Jun. 19, 2007 (US) 60936284

(51) Int. Cl. C09D 5/16; C12Q 1/56; C12Q 1/37; C12Q 1/48; C08B 37/00; C07D 311/06; C08B 37/10; C08B 37/02; C07C 55/22; C07C 229/04; C07C 65/10

(52) U.S. Cl. 106/18.32; 435/13; 435/23; 435/15; 106/15.05; 536/53; 549/399; 536/21; 536/51; 549/400; 562/590; 562/566; 562/564; 560/143

(57) ABSTRACT

Methods and compositions are provided for the reduction of fouling of objects present in marine environments. The methods and compositions include anticoagulants, such as, for example, glycosaminoglycans, coumarin-type molecules, metal chelators, plasminogen activators and platelet inhibitors. The methods include reducing marine fouling, comprising incorporating an anticoagulant compound into a marine coating. In addition, the methods include identifying compounds useful for reducing marine fouling, comprising measuring either blood coagulation or barnacle cement polymerization in the presence and absence of the compound, wherein a reduction in the blood coagulation or the barnacle cement polymerization in the presence of the compound identifies the compound as useful for reducing marine fouling. The coagulation or the polymerization can be measured by a serine protease activity or a transglutaminase activity.

### RELATED APPLICATIONS

This application claims priority to U.S. Provisional Patent Application No. 60/936,284, filed Jun. 19, 2007, the entire contents of which are hereby incorporated by reference.

### GOVERNMENT INTEREST

This presently disclosed subject matter was made with U.S. Government support under Grant Nos. N00014-05-1-0469, N00014-05-1-0580, N00014-06-WX-20770, N00014-07-WX-20504, N00167-03-M-0345 and N00167-04-M-0214 awarded by the U.S. Office of Naval Research. Thus, the U.S. Government has certain rights in the presently disclosed subject matter.

### TECHNICAL FIELD

The presently disclosed subject matter pertains to the use of anticoagulants as antifouling agents for marine applications.

### BACKGROUND

Fouling, the settlement and growth of organisms on man-made objects, has long been a concern of mariners, militaries and merchants. Any inert object that is placed in the sea will be colonized by marine fouling organisms within days to weeks, depending on environmental conditions. Of practical concern is the fouling of structures such as off-shore platforms and aquaculture facilities (weighed down by fouling organisms), power plant cooling systems (blocked by fouling organisms) and most notably ship hulls. The fouling of ship hulls leads to a drastic reduction in performance and fuel efficiency. Fouling can also interfere with acoustic and other underwater instrumentation. The fouling of ship hulls costs the Defense and shipping industries billions of dollars every year.

Since ancient times, mariners have fashioned solutions to the problem of fouling. Antifouling coatings date back at least to the ancient Romans who would pound copper into thin sheets to coat the hulls of their ships (W.H.O.I. 1951). The effectiveness of copper coatings is a result of the toxicity of copper ions to settling larvae. Heavy metals based coatings, have been, and still are the most effective form of antifouling coating.

In the 1960s a new form of heavy metal based antifouling coatings was developed which incorporated tributyltin compounds. These organotin coatings are broad spectrum biocides and are extremely effective because they kill fouling larvae. Naval and commercial ships world-wide were coated with tin-based paints. Unfortunately, leaching of broad spectrum biocidal tin compounds into the water devastated populations of non-target species can result. In mollusks, exposure of non-lethal levels of tin leads to imposex, the development of male characteristics (Smith 1981) and behavioral castration (Straw & Rittschof 2004) in females. The environmental concern over organotin-based antifouling paints was so extreme that paint companies voluntarily withdrew tributyltin based paints from the Global market in 2003 and a world-wide ban on its use was imposed.

Today, the majority of large ships continue to use heavy metal-based paints, primarily in the form of copper. Although the toxic effects of copper to non-target species are not as severe as those of tin, their use is not ideal. The emphasis of new research on marine coatings is on foul-release coatings. Foul-release coatings are primarily composed of relatively low toxicity silicone. Silicone coatings allow fouling organisms to settle, but prevent firm attachment. Silicone coatings show potential as marine coatings, although their mechanisms of action are not fully understood.

Barnacles are one of the most common and dominant members of marine fouling communities. The biochemical mechanism by which barnacle cement polymerizes is poorly understood. There is some evidence that proteolytic enzymes (Dougherty 1996, 1997) and salinity (Nakano et al. 2007) play a role in cement polymerization. The details of these mechanisms, however, have not been fully explored. Initial biochemical investigations into the nature of barnacle cement were thwarted by its inherent insolubility. Creative techniques developed to obtain liquid cement (Walker 1972; Cheung et al. 1977) and denatured solidified cement (Barnes and Blackstock 1976, Yan and Pan 1981, Naldrett 1993, Kamino et al. 1996, Kamino 2001) have allowed for compositional analysis. Barnacle cement is composed of 90% protein (Walker 1972, Naldrett 1993) and is an aggregate of at least ten major proteins (Naldrett and Kaplan 1997, Kamino 2006). Some, but not all, of the barnacle cement proteins have been isolated and sequenced (reviewed in Kamino 2006, 2008). In part, chemical stability of polymerized barnacle cement is achieved through cysteine cross-links and hydrophobic interactions (Barnes and Blackstock 1976, Naldrett and Kaplan 1997, Kamino et al. 2000).

Polymerization of barnacle cement proteins occurs via cysteine cross-links and hydrophobic interactions (Barnes & Blackstock 1976; Naldrett 1993; Kamino et al. 2000). The importance of cysteine cross-links to barnacle cement stability is shown by its solubility in reducing agents, such as beta-mercaptoethanol, which break cysteine cross-links (Barnes & Blackstock 1976; Yan 1981; Naldrett 1993). Solubility of cement in beta-mercaptoethanol varies between species (Naldrett 1993). Species showing the highest levels of cysteine show the highest resistance to beta-mercaptoethanol.

Few studies have focused specifically on the enzymes involved in barnacle cement polymerization. Dougherty (1996; 1997) considered protease activity in unpolymerized cement of the barnacle Chthamalus fragilis using a FTC-casein substrate. Proteolytic cleavage of cement proteins may alter protein conformation so as to allow the formation of disulfide bonds, thereby facilitating polymerization. Protease activity was shown in C. fragilis cement and activity was enhanced in the presence of calcium ions. Using protease inhibitors and dye-labeled PepTag peptides, Dougherty (1996; 1997) showed the activity of zinc metalloprotease with a preference for carboxyl-terminal basic amino acids.

There remains a long-felt need for additional antifouling agents in marine application, particularly antifouling agents without toxic effects to non-target species.

### SUMMARY

The presently disclosed subject matter provides processes and compositions to inhibit the fouling of objects placed in a marine environment. In some embodiments, the foul-release processes and compositions disclosed herein pertain to the inhibition of polymerization of barnacle cement. In some embodiments, a process is provided for reducing marine fouling, comprising incorporating an anticoagulant other than silicone into a marine coating. In some embodiments, a process is provided for inhibiting the fouling of an object in a marine environment which comprises using an anticoagulant other than silicon to inhibit polymerization of barnacle cement such that the ability of the barnacle to adhere to the substrate is lessoned. In some embodiments, a process is provided for inhibiting the fouling of an object in a marine environment, which comprises forming on the object, before exposure to the environment, a coating comprising an anticoagulant other than silicon.

In some embodiments, the anticoagulant is selected from the group including, but not limited to, glycosaminoglycans (including molecules such as heparin sulfate and dextran sulfate), coumarin-type molecules (including molecules such as DICOUMAROL and WARFARIN), metal chelators (including molecules such as EDTA, EGTA and citrate), plasminogen activators (including molecules such as tissue plasminogen activator) and platelet inhibitors (including molecules such as aspirin).

In some embodiments of the presently disclosed subject matter, a method is provided of identifying compounds useful for reducing marine fouling comprising, measuring either blood coagulation or barnacle cement polymerization in the presence and absence of the compound, wherein a reduction in the blood coagulation or the barnacle cement polymerization in the presence of the compound identifies it as useful for reducing marine fouling. In some embodiments, the coagulation or the polymerization is measured by measuring a serine protease activity. In some embodiments, the serine protease is a trypsin-like serine protease. In some embodiments, the coagulation or the polymerization is measured by measuring transglutaminase activity.

In some embodiments of the presently disclosed subject matter, a process is provided for reducing marine fouling, comprising incorporating the identified compound into a marine coating.

In some embodiments of the presently disclosed subject matter, a process is provided for inhibiting the fouling of an object in a marine environment, comprising using the identified compound to inhibit polymerization of barnacle cement such that the ability of the barnacle to adhere to the object is lessoned.

In some embodiments of the presently disclosed subject matter, a marine foul-release coating composition is provided comprising the identified compound.

Accordingly, it is an object of the presently disclosed subject matter to provide methods and compositions for reducing marine fouling. This and other objects are achieved in whole or in part by the presently disclosed subject matter. An object of the presently disclosed subject matter having been stated above, other objects and advantages will become apparent upon a review of the following description.

### BRIEF DESCRIPTION OF THE DRAWINGS

FIGS. 1A-1B. Light microscope photographs of the adhesive plaque (baseplate with cement layer) of two barnacles (Balanus amphitrite) grown on a VIRIDIAN silicone coated glass plate at the Duke University Marine Laboratory. The barnacle on the left (A) expresses the thin, hard phenotype, whereas the barnacle on the right (B) expresses the thick, gummy phenotype. Note the radial canals which can be seen on the thin, hard plaque barnacle but not on the thick, gummy plaque barnacle.

FIGS. 2A-2D. Comparison of a fibrin blood clot and barnacle cement using scanning electron microscopy (SEM) and atomic force microscopy (AFM). A) SEM image of a fibrin blood clot. Cross linked fibrin, red blood cells and platelets are shown in the image. Photo credit: Yuri Veklich and John W. Weisel, University of Pennsylvania School of Medicine. B) AFM image of barnacle cement left on a glass slide. A barnacle was removed from a silicone substrate and allowed to reattach to a glass slide. After two days in seawater, the barnacle was removed and the residual cement was imaged. C) Confocal light microscope image of a fibrin blood clot, prepared by adding calcium and thrombin to isolated plasma (Collet et al. 2005). D) AFM image of a droplet of barnacle cement cured in seawater. One μl of liquid barnacle cement was obtained and deposited on a glass slide. The droplet was immediately covered by another slide and placed in seawater for 2 days.

FIG. 3. Fourier Transform Infra Red spectroscopy (FTIR) spectra of a fibrin blood clot (top) and of polymerized barnacle cement (bottom). The amide I, II and III region is shown (950-1800 cm⁻¹). Significant peaks are labeled with wavenumbers. The FTIR spectrum of polymerized barnacle cement is very similar in both peak position and relative peak intensity to that of clotted fibrin, indicating that the protein configuration and secondary structure of barnacle cement is similar to that of clotted fibrin. The FTIR spectrum of barnacle cement was obtained from residual cement left by whole barnacles on an ATR crystal. The FTIR spectrum of fibrin is from Bramanti et al. 1997.

FIGS. 4A-4B. Western blotted PVDF membrane of barnacle cement proteins immunostained for trypsin. A) Lanes A, B & C are barnacle cement, lane D is a trypsin positive control (4 μg bovine trypsin), and lane E is molecular weight markers (a mix of 10 proteins, 10-225 kDa). Positive staining is observed as dark horizontal bands. Staining in the positive control lane appears primarily at 24 kDa. No staining is observed for molecular weight markers. Positive staining in barnacle cement lanes occur at 90 kDa. In lane C, both the inactive (higher arrow) and activated (lower arrow) forms of the enzyme can be seen. B) Western blotted cement proteins (WB) are shown next to SDS-PAGE (reducing conditions) of cement proteins (BC) and molecular weight markers (MW), labeled in kDa. Trypsin immunogen is from bovine pancreas. Immunostaining of barnacle cement for trypsin, a key enzyme responsible for blood coagulation as shown in FIG. 4B, has yielded consistent and reproducible staining at 90 kDa. The staining at 90 kDa indicates a trypsin-like molecule is present in barnacle cement and that the polymerization of barnacle cement occurs by a similar enzymatic mechanism to that of blood coagulation.

FIG. 5. Immunoblot of a fibrinogen-like protein in barnacle cement. Fibrinogen is the major structural protein that comprises a vertebrate blood clot. Positive staining is observed in both dot blots of whole cement droplets (right) and barnacle cement separated using non-denaturing gel electrophoresis and Western Blotted onto a PVDF membrane (left). The quantity of fibrinogen like protein in barnacle cement is roughly 0.5 mg/ml. This estimate is based on a comparison of the staining intensity of cement dot blots to that of controls.

FIG. 6. Protein profile for barnacle cement polymerized in the presence of distilled water (control) or one of 5 anticoagulants heparin, warfarin, trypsin inhibitor, EGTA or EDTA. Each peak represents an individual protein. For each treatment, 2 μl of either distilled water or anticoagulant was added to 1 μl unpolymerized cement taken from a thick, gummy phenotype barnacle. Polymerization was allowed to proceed for 2 minutes. Samples were analyzed with SDS-PAGE using a 4-20% acrylamide gel. The gel was stained with Coomassie Blue and analyzed using Scion Image. The number and intensity of proteins is significantly higher in all five anticoagulant treatments, indicating that all five anticoagulants effectively inhibit the polymerization of barnacle cement. The difference between deionized water and anticoagulant treatments is particularly noticeable in the 50-150 kDa region.

FIGS. 7A-7C. GC traces with Mass Spec identification for commercially available silicone foul-release coatings. The traces show that cyclic silicone monomers are being released from the surfaces of the coatings. A) Methanol extraction was performed on VERIDIAN coated glass panels that had been used for barnacle settlement for two years. Each peak on the trace represents a molecule released from the silicone surface. Peaks with matches in the NIST Mass Spec library are labeled with molecular names and structure where available. B) GC Trace with mass spectrometry identification for Dow Corning Silastic T2® silicone, extracted with methanol. Methanol extraction was performed on silicone coated glass panels that had been used as barnacle substrates for two years. The name and structure (if available) of extracted molecules identified in the NIST database are noted. Each peak on the trace represents a molecule released from the silicone surface. The GC/Mass Spec trace provides evidence that silicone monomers are being released from the surface and would therefore be available to interfere with the polymerization of barnacle cement. C) Mass spec traces for GC peaks shown in (A) and (B) with matching NIST mass spectral library traces.

FIGS. 8A-8D. Measurements of barnacle initial- and reattachment-removal forces. A) Bar graph of the initial removal force of barnacles removed from T2 (black bars) and reattachment removal force (gray bars). Barnacles were reattached to glass panels coated with 0 (control), 0.1, 1.0 or 10.0 mg ml⁻¹ heparin for 7 days. Removal force decreased for all barnacles on heparin treatments. A highly significant difference from initial removal force was shown for 10 mg ml⁻¹ heparin (paired samples t-test: p=0.017). B) Mean removal force (±SE) for barnacles reattached to deionized water (dH₂O), sucrose (1 mg ml⁻¹) or heparin (1 mg ml⁻¹) coated T2 silicone over time. The same group of barnacles was successively reattached to the same treatment for 1, 2, 3 and 4 days (in that order); n=10 barnacles for dH₂O and sucrose, n=10 for heparin 1 day, and n=9 for heparin 2, 3 and 4 days. The heparin coated results show a significant difference from the sucrose results for 1 day reattachment (pre-planned sequential Bonferroni pairwise comparison: p<0.05). Statistical comparisons were not conducted on 2, 3, and 4 day trials as there is a potential for the results to be influenced by previous reattachment trials. C) Mean removal force (±SE) for barnacles reattached to clean glass (marked as 0.00) and glass coated with heparin at three concentrations. Initial removal force from T2 silicone is shown for comparison. The symbol “*” indicates a significant difference from the deionized water control group (pre-planned sequential Bonferroni pairwise comparison: p<0.05). D) Data were fitted with a sigmoidal dose-response curve to calculate EC₅₀, r²=0.61. Barnacles were reattached for 7 days; n=5 barnacles for each concentration.

FIGS. 9A-9J. Optical microscope and AFM images of barnacle cement left on class. A) Flat (left panel) and 3D projected (right panel) AFM images of residual barnacle cement on glass. A barnacle was removed from a silicone substrate and allowed to reattach to a clean glass slide in seawater for 2 days. The barnacle was then removed with a sharp probe and the residual cement imaged. B) Flat (left panel) and 3D projected (right panel) AFM images of barnacle cement left on heparin coated glass. A barnacle was removed from a silicone substrate and allowed to reattach to a glass slide coated with 10 mg ml⁻¹ heparin for 1 week in seawater. The barnacle was removed with a sharp probe and the residual cement imaged. Optical microscope images of residual cement left by barnacles reattaching for one week to: C) clean glass; D) heparin coated glass; E) clean class; F) BSA coated glass (1 mg ml⁻¹); G) BSA coated glass (10 mg ml⁻¹); H) heparin coated glass (1 mg ml⁻¹); and I) & J) heparin coated glass (10 mg ml⁻¹).

FIG. 10. SDS-PAGE of unpolymerized barnacle glue, unpolymerized barnacle glue plus heparin, and molecular weight standards. A precast, 4-20% acrylamide gel was used and stained with Coomassie Blue. Note that the bands smaller than 31 kDa, which correspond to serine proteases and their peptide products, are present for glue only but do not appear when heparin is added. In addition, the number and intensity of bands between 115-85 kDa is increased when heparin is added.

FIG. 11. Mean transglutaminase activity (±SE) for unpolymerized barnacle cement. The assay was conducted using a cadaverine coated 96-well plate; n=19 barnacles. The mean and SEM for the blank (no enzyme present) and positive control are shown for comparison.

### DETAILED DESCRIPTION

In accordance with the presently disclosed subject matter, processes and compositions are provided for reducing marine fouling. Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety. The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

While the following terms are believed to be well understood by one of ordinary skill in the art, the following definitions are set forth to facilitate explanation of the presently disclosed subject matter.

Following long-standing patent law convention, the terms “a” and “an” mean “one or more” when used in this application, including the claims.

As used herein, the term “anticoagulant” means any ion, molecule, element, chemical or compound, or any substance comprising such an ion molecule, element, chemical or compound, that interferes, directly or indirectly, with the function of the proteolytic enzymes involved in blood coagulation.

The terms “chemical” “compound” and “molecule” are herein used interchangeably.

As used herein, the term “object” is not meant to be limited in any way, and represents anything attached or free standing that may be present in a marine environment.

As used herein, the terms “reduce”, “decrease” and “inhibit” are used interchangeably and refer to an activity whereby marine fouling and/or polymerization of barnacle cement is reduced below that observed in the absence of a composition of the presently disclosed subject matter.

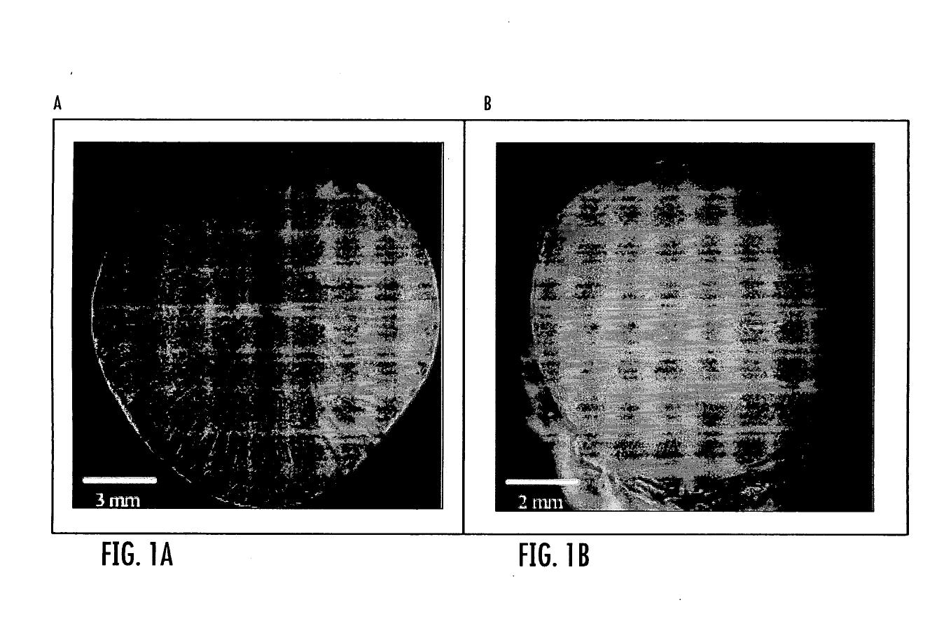

Researchers working with barnacles on silicone foul-release coatings have noted variation between individual barnacles in the morphology of the cement the animals use to adhere to the silicone substrate (Berglin & Gatenholm 2003; Wiegemann & Watermann 2003; Holm et al. 2005). Barnacles grown on surfaces composed of long chain hydrocarbons produce thin, hard cement. In contrast, barnacles grown on silicone-based coatings sometimes produce thick, gummy cement. It is pointed out in Example 1 and FIG. 1, that radial canals can be seen on the thin, hard plaque barnacle (FIG. 1A) but not on the thick, gummy plaque barnacle (FIG. 1B). The proportion of individual barnacles with thick, gummy cement varies with the type of silicone surface used. The propensity to develop thick, gummy cement has been shown to be heritable by Holm et al. (2005). The generation of thick, gummy cement rather than thin, hard cement is indicative of a defect in the process of cement polymerization.

Jaques et al. (1946) discovered that blood in contact with methyl-chloro-silane (silicone) coated beakers does not clot. Silicone can release free silicone monomers that interfere with the enzymes that cause blood coagulation, resulting in inhibition of blood clot formation. The formation of a stable fibrin blood clot in vertebrates is generated by two closely interrelated cascades of serine proteases (Davie & Fujikawa 1975). The overall product of these cascades is a network of cross-linked fibrin monomers. In the blood clotting process, the product of one reaction is the substrate for the next reaction (Davie & Rantoff 1964; Macfarlane 1964; Davie 1986). Enzymes are synthesized as inactive precursors (zymogens) and converted to active forms by selective enzymatic cleavage of peptide bonds. The overall product of these proteolytic cascades is the amplification of a small stimulus (an injury) into a physiological response (a blood clot) (Neurath & Walsh 1976; Neurath 1986). This system is efficient and easily regulated. Although the two pathways involved in blood clotting in vertebrates are complementary, deficiency of a single factor in either pathway can prevent blood from clotting.

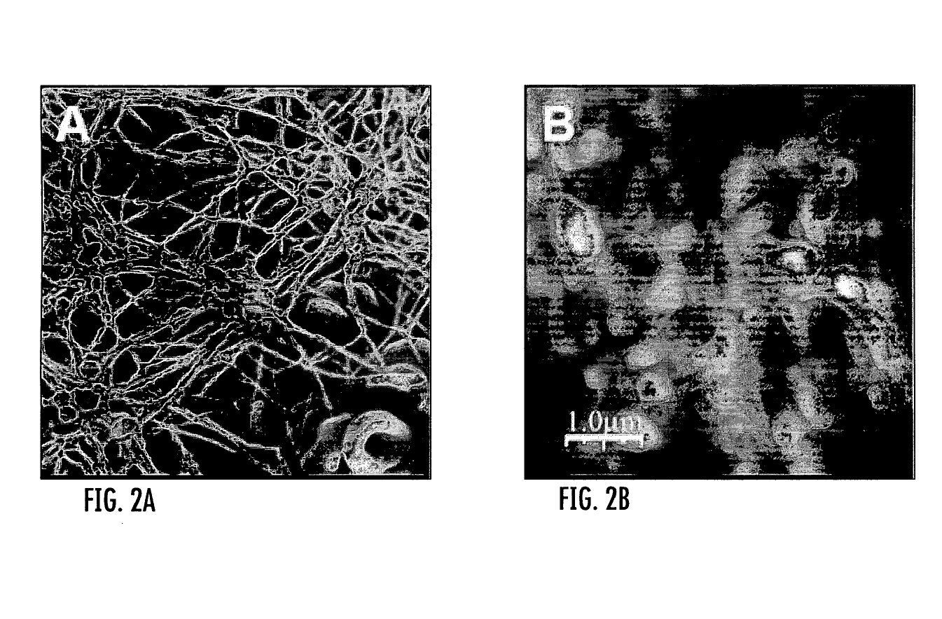

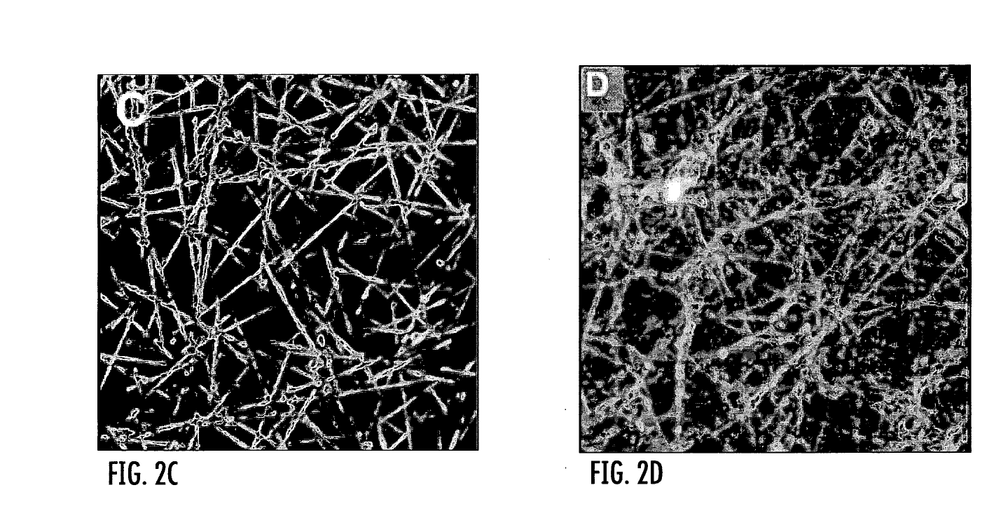

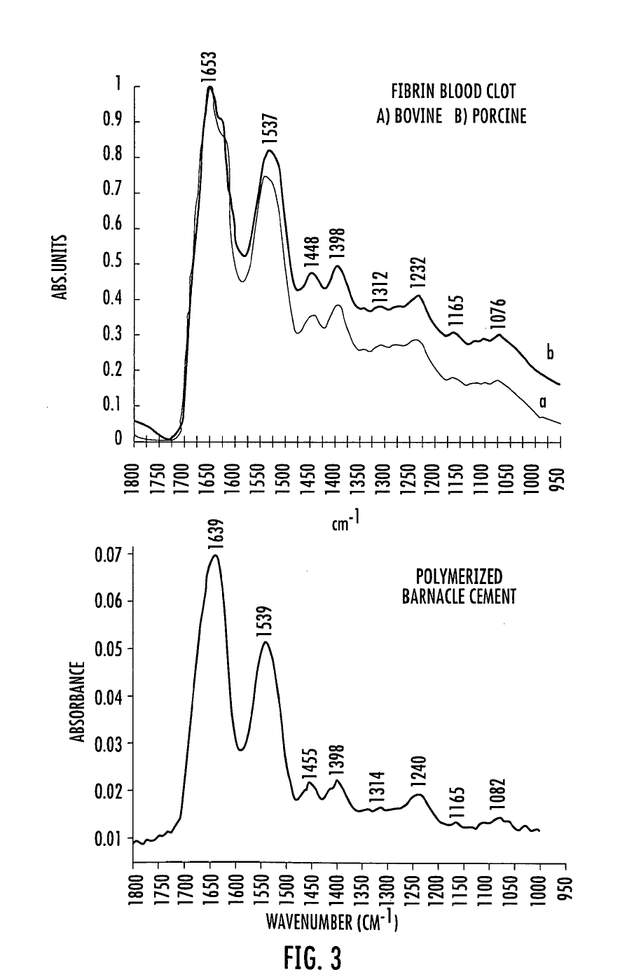

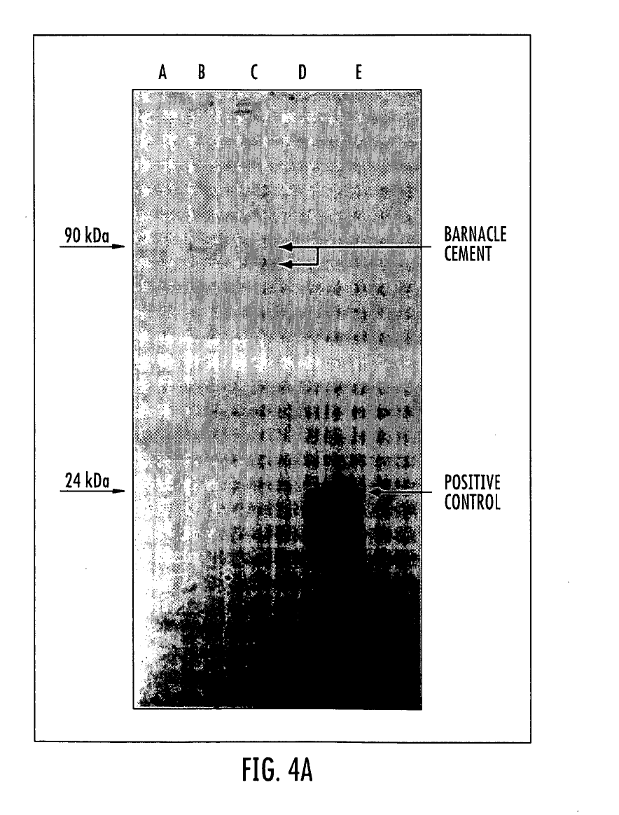

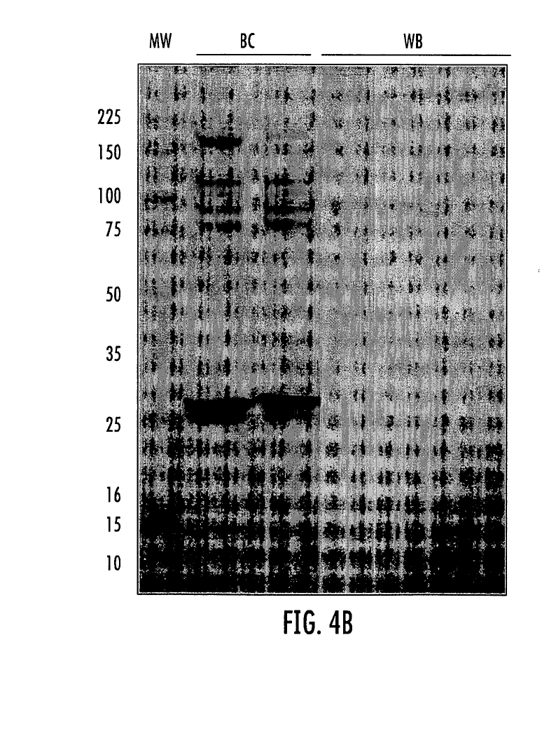

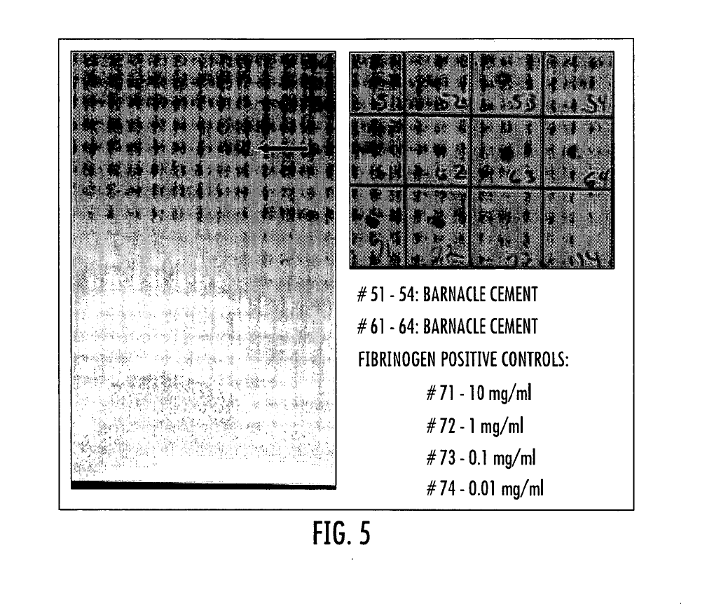

The observation that silicone can act as a blood anticoagulant and can also prevent barnacle cement hardening resulted in the discovery of the presently disclosed subject matter. Specifically, the presently disclosed subject matter demonstrates that blood coagulation and barnacle cement polymerization occur by a similar enzymatic mechanism. In addition to showing that similar to blood, barnacle cement does not coagulate in the presence of silicone, the present disclosure provides evolutionary and biochemical studies demonstrating that the processes involved in blood coagulation and barnacle cement polymerization are similar (see Examples 1-11; FIGS. 1-11). For example, atomic force microscopy (AFM) and infrared spectroscopy (FTIR) reveal a striking structural similarity between clotted fibrin and polymerized barnacle cement (see FIGS. 2A-2D; FIG. 3; Examples 2-4). As shown in FIG. 2, polymerized barnacle cement appears to be composed of a network of closely interlocking fibrous proteins similar to clotted fibrin. In addition, there is evolutionary evidence for the conservation of the anticoagulation mechanism (see Example 3) and the FTIR spectrum shown in FIG. 3 of polymerized barnacle cement is nearly identical to the FTIR spectra of bovine and porcine fibrin blood clots (see Example 4). Further, the major components of the blood clotting system, trypsin and fibrinogen, are present in barnacle cement demonstrated using immunostaining (see FIGS. 4A-4B; FIG. 5; Example 5).

Accordingly, in some embodiments of the presently disclosed subject matter, the potential is demonstrated that blood coagulation and barnacle cement polymerization occur by a similar enzymatic mechanism. For example, barnacles which have a defect in cement polymerization have a reduced ability to adhere to a substrate. As barnacles often serve as a substrate for less tenacious species, a decrease in the number of barnacles would have a significant effect on overall marine fouling. Therefore, in some embodiments, the presently disclosed subject matter provides chemicals that can prevent the coagulation of blood (anticoagulants) to prevent the polymerization of barnacle cement. Accordingly, in some embodiments, the presently disclosed subject matter describes anticoagulant chemicals as useful agents for reducing marine fouling. In some embodiments, the presently disclosed subject matter provides methods for identifying useful compounds for reducing marine fouling by screening potential compounds for the ability to inhibit and/or reduce one or both of blood coagulation and barnacle cement polymerization.

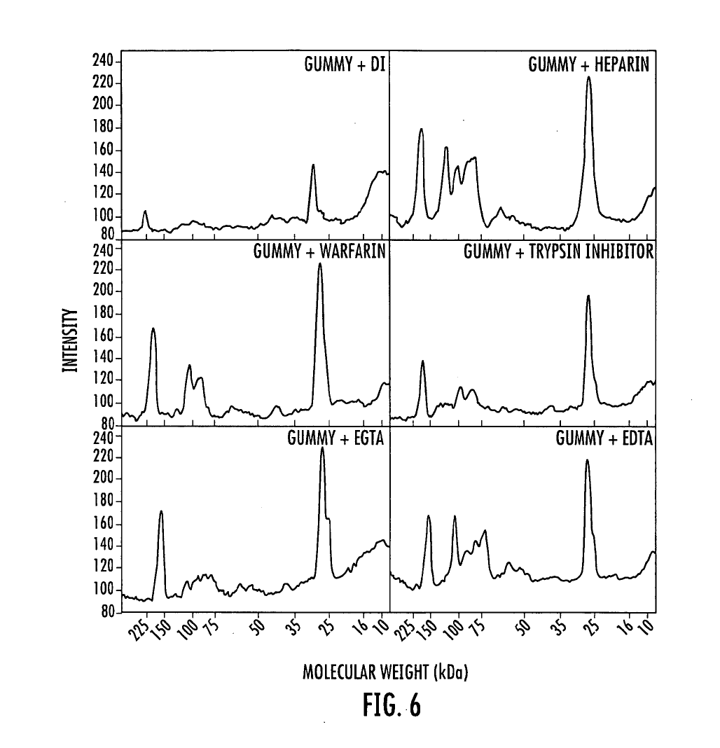

The following Examples and Figures demonstrate the ability of anticoagulants to inhibit polymerization of barnacle cement. The protein profile for barnacle cement polymerized in the presence of distilled water (control) or one of 5 anticoagulants (heparin, warfarin, trypsin inhibitor, EGTA or EDTA) is shown in FIG. 6 (see also Example 6). The number and intensity of proteins is significantly higher in all five anticoagulant treatments, indicating that all five anticoagulants effectively inhibit the polymerization of barnacle cement.

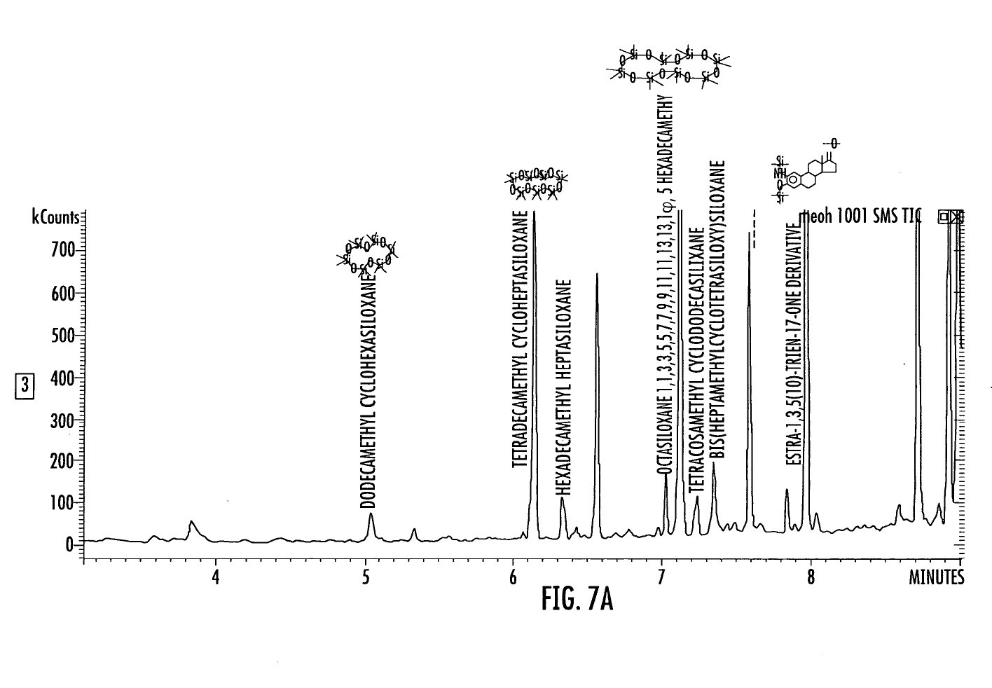

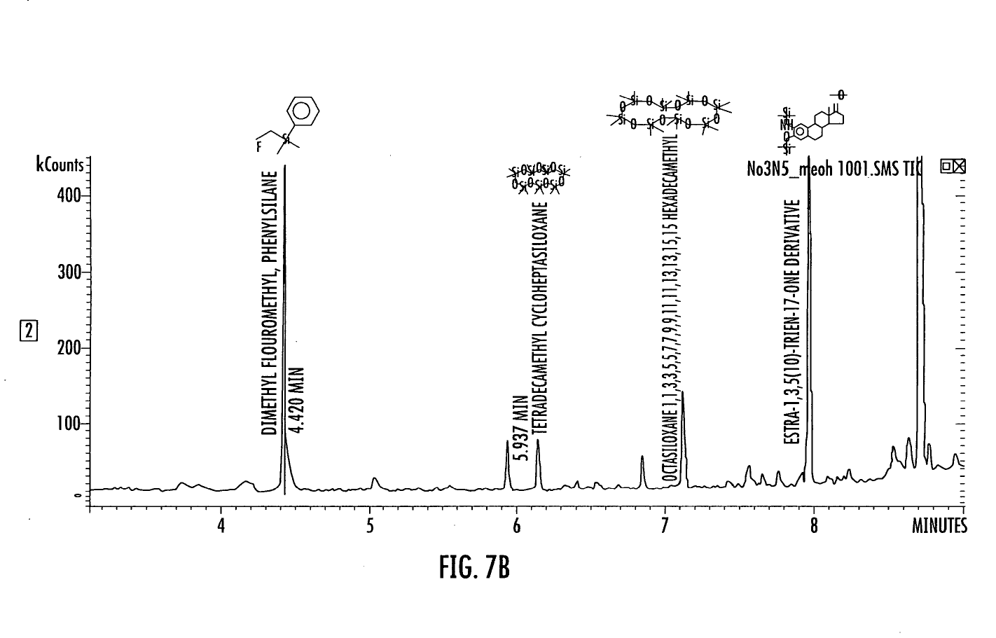

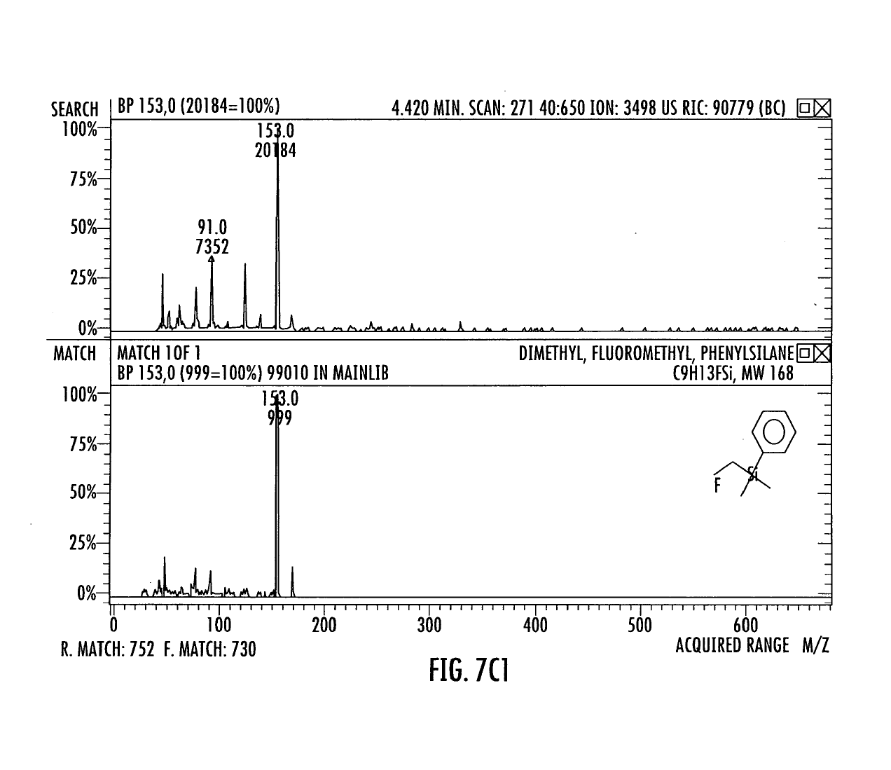

The present inventors have also demonstrated that silicone monomers are released from the surface of VERIDIAN, a commercially available silicone foul-release coating (see FIGS. 7A-C; Example 7). The silicone monomers are available to interfere with the polymerization of barnacle cement. Silicone monomers act as an effective anticoagulant of both blood and barnacle cement. In preventing cement polymerization, silicone monomers cause the thick, gummy cement that is observed on 30% of barnacles settled on silicone surfaces.

Medical research on blood coagulation has led to the identification of a large number of anticoagulants. The mechanism of action for each of these anticoagulants has been well studied and each anticoagulant targets the blood coagulation system in a different way. Common drugs used as anticoagulants include glycosaminoglycans (including, but not limited to, heparin sulfate, dextran sulfate), coumarin drugs (including, but not limited to, DICOUMAROL, WARFARIN), metal chelators (including, but not limited to, EDTA, EGTA, citrate) and platelet inhibitors (including, but not limited to, aspirin), among others. Heparin, which is produced naturally by the body, functions by activating antithrombin III (reviewed in Capila and Linhardt 2002). Antithrombin III is a serine protease inhibitor that prevents the activity Of thrombin and factor Xa, thereby preventing the formation of a fibrin clot. Heparin also binds Ca²⁺ ions (Nieduszynski 1989, Landt et al. 1994, Rabenstein et al. 1995, Karpukhin et al. 2006), which are essential to serine protease and transglutaminase activity. For thrombin and factors VII, IX, and X, y-carboxylation of glutamic acid residues during synthesis is necessary for Ca²⁺ binding. Coumarin drugs inhibit the recycling of vitamin K, an essential cofactor to y-carboxylation (Ansell et al. 2004). Metal chelators bind Ca²⁺, which limits availability of Ca²⁺ to enzymes. Binding of Ca²⁺ to blood coagulation factors VII, IX, XI, X, XIII and thrombin is essential to their active conformation (Davie and Fujikawa 1975). Lastly, aspirin inhibits the activation of platelets, which prevents the formation of a platelet plug, a precursor to a vertebrate fibrin clot (Szczeklik et al. 1992).

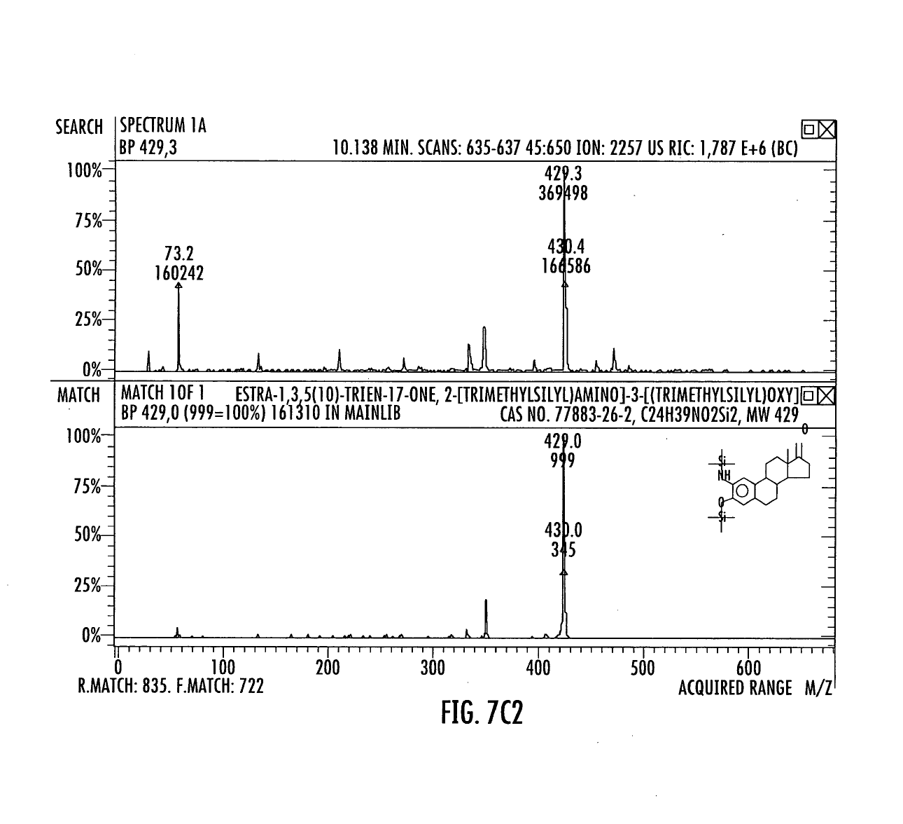

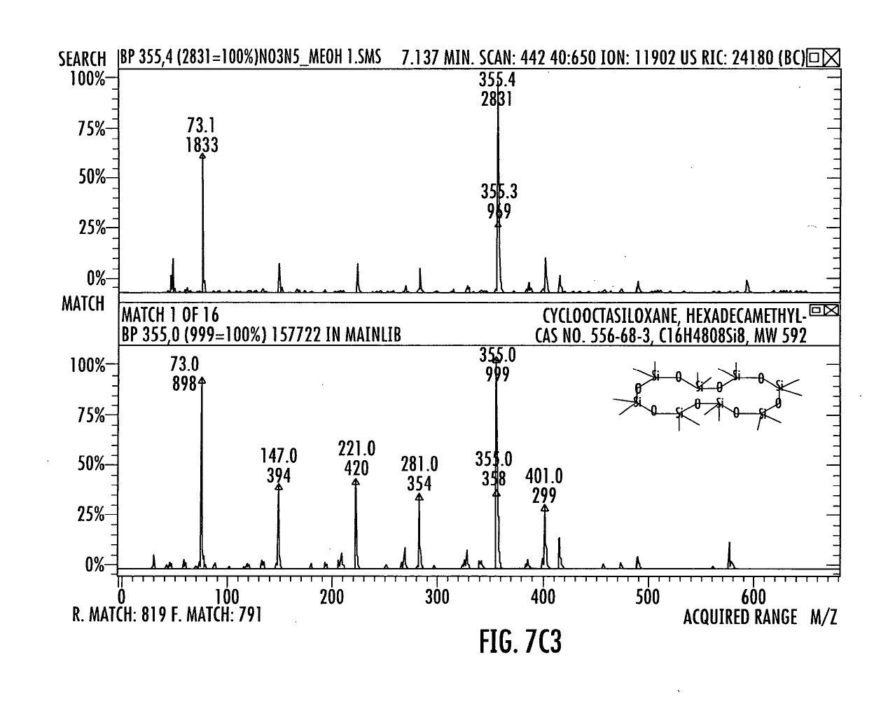

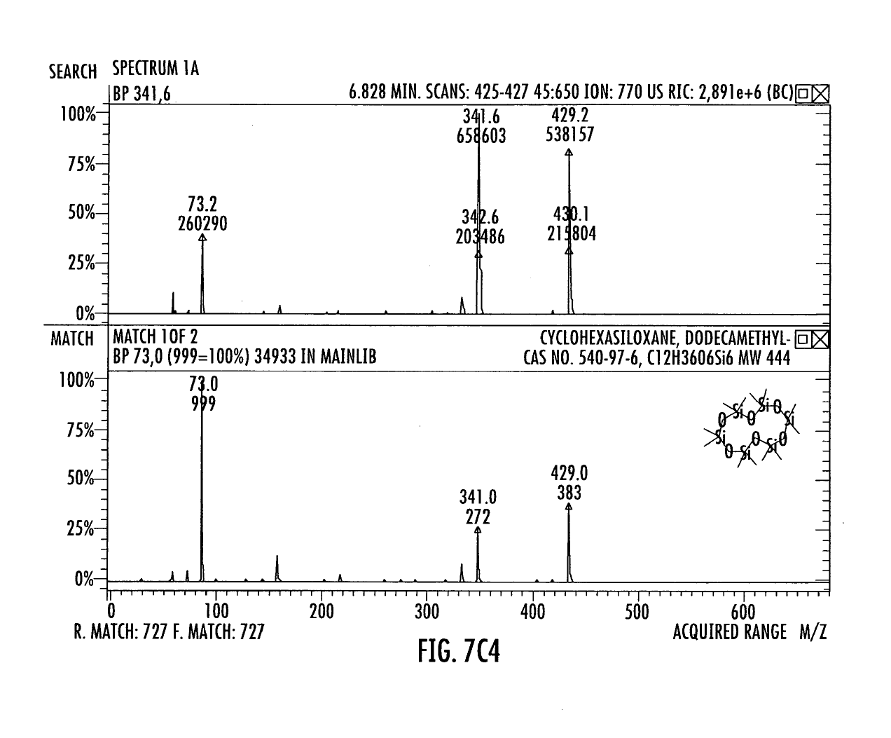

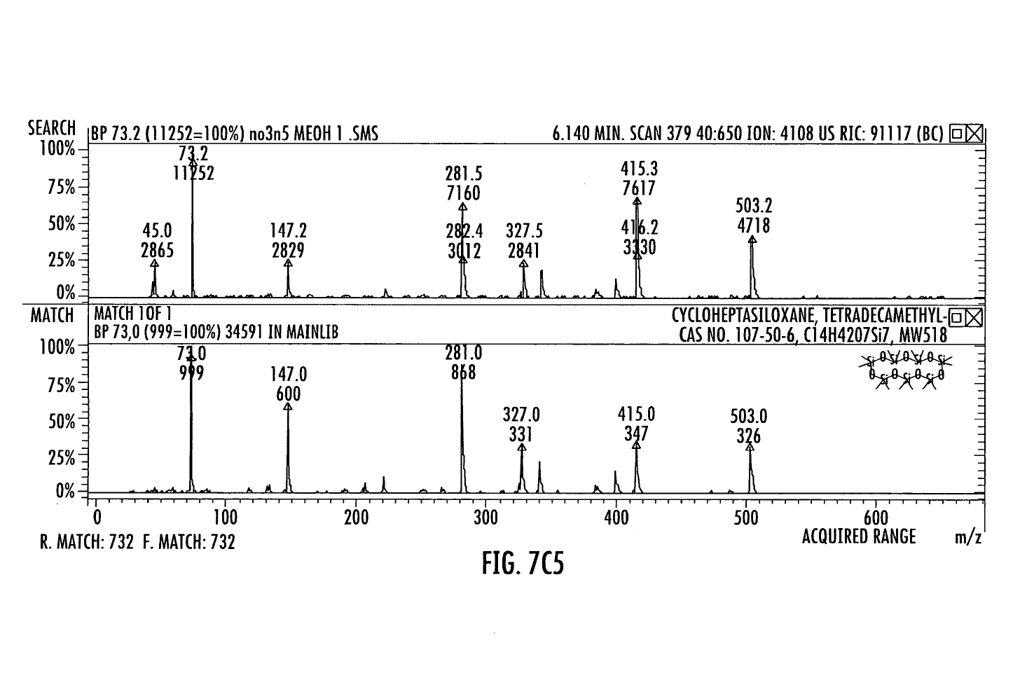

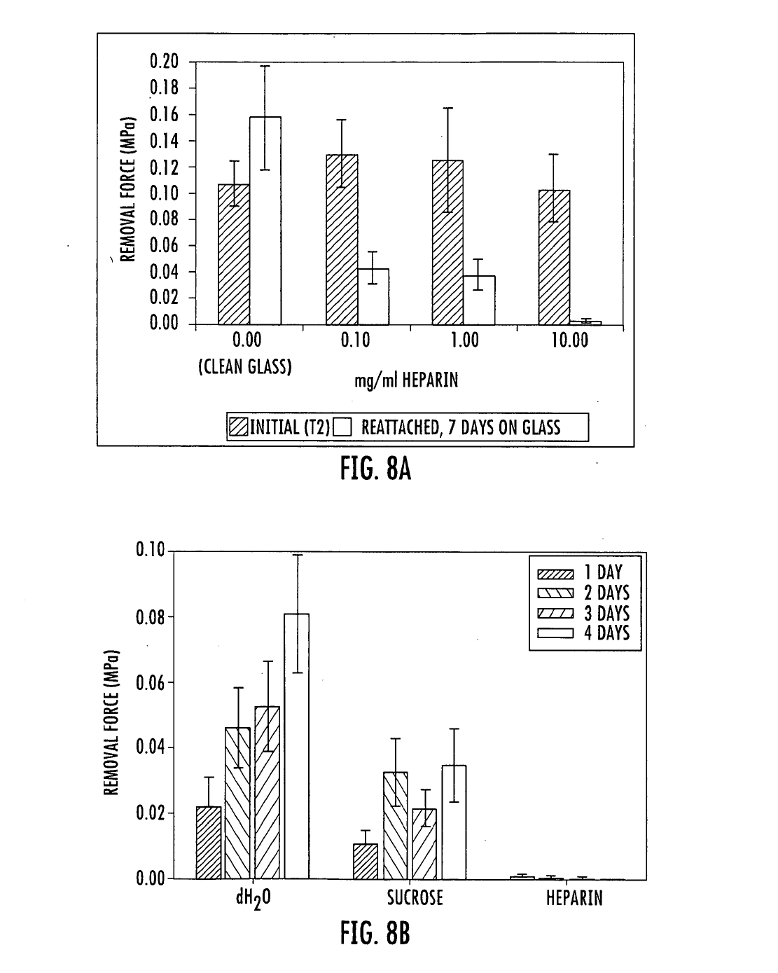

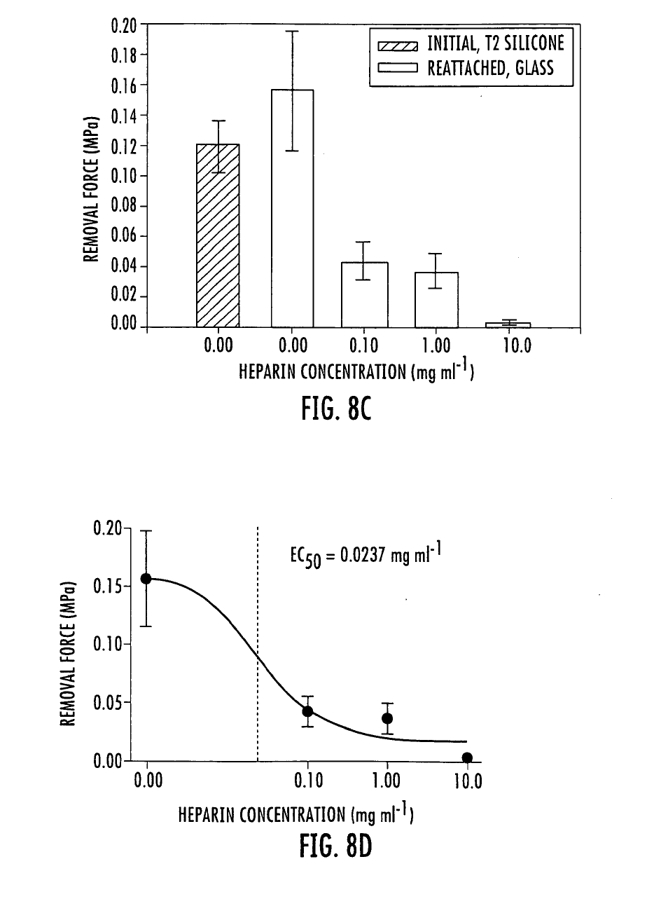

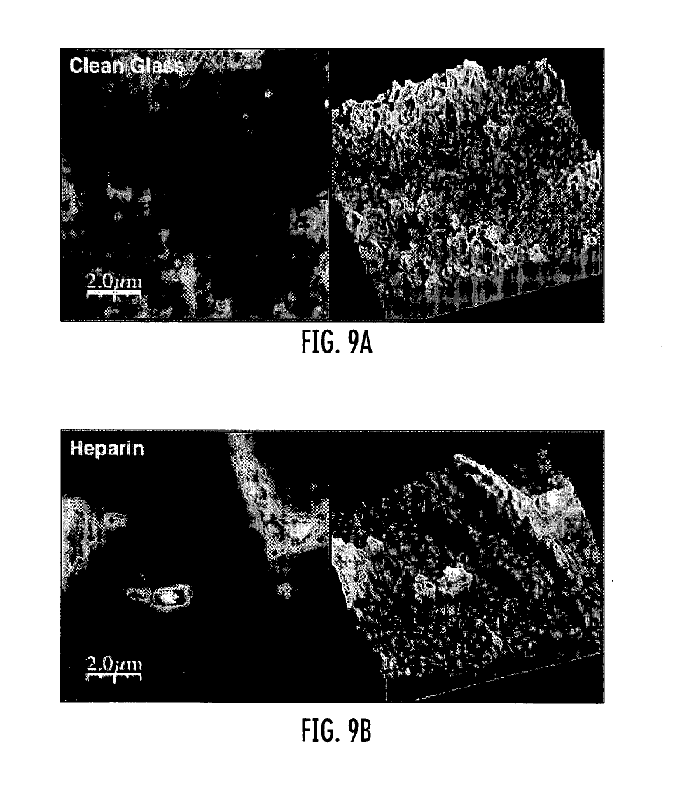

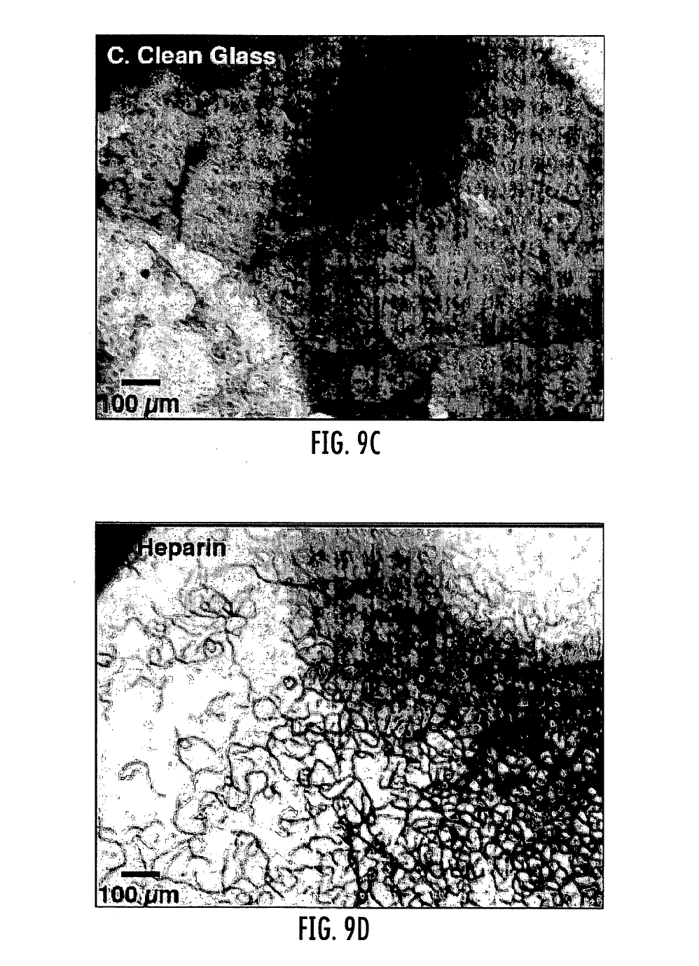

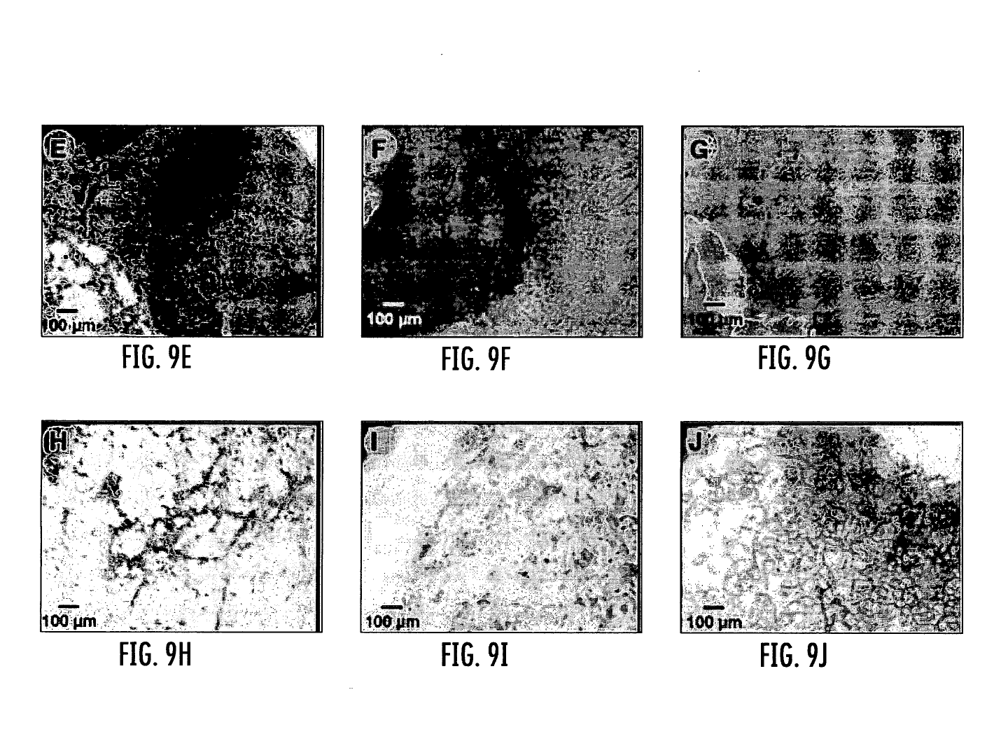

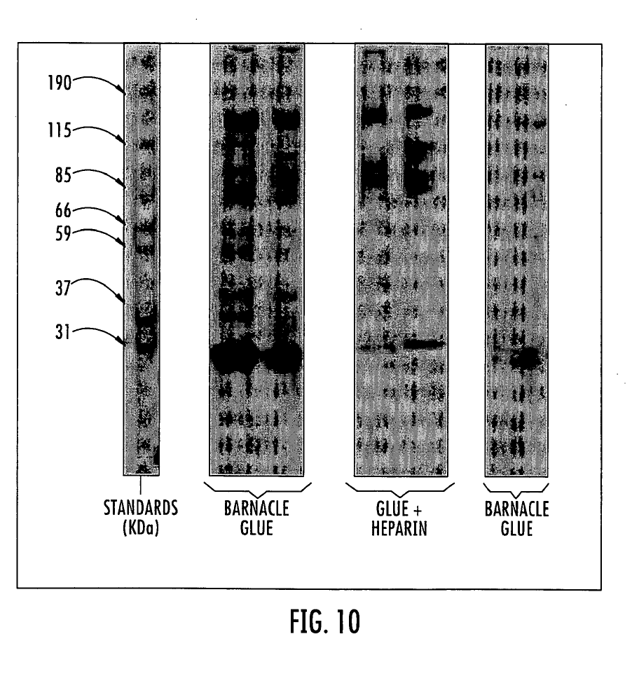

The ability of anticoagulant compounds to inhibit barnacle cement polymerization is further demonstrated in the following Examples and Figures. The initial removal force and reattachment removal force of barnacles is decreased when the barnacles are grown on substrates treated with the anticoagulant heparin (see FIG. 8; Example 8). Optical light microscopy and atomic force microscopy were used to show that residual barnacle cement left on clean glass appears as a dense network of interweaving fibers, whereas residual cement left on heparin coated glass appeared as longer fibers with minimal networking (see FIGS. 9A-9J; Example 9). SDS-PAGE experiments demonstrated that the protein signature of barnacle cement is significantly different in the presence of heparin (see FIG. 10; Example 10). The analyses have shown that barnacle cement is composed of proteins corresponding to the molecular weight of the serine proteases found to be active in cement polymerization, and the peptides produced by cleavage of the serine proteases. This result provides further evidence that heparin is interfering with the cascade of serine proteases active in barnacle cement polymerization.

Therefore, the presently disclosed subject matter provides evidence that polymerization of barnacle cement occurs by a similar enzymatic mechanism to that of blood coagulation. As a result, chemicals capable of preventing the coagulation of blood (anticoagulants) can also prevent the polymerization of barnacle cement. In some embodiments of the presently disclosed subject matter, anticoagulant chemicals can be incorporated as additives in foul-release coatings to reduce or alleviate the problem of marine fouling by inhibiting the polymerization of barnacle cement. Inhibiting barnacle cement polymerization lessons a barnacle's ability to adhere. As barnacles often serve as a substrate for less tenacious species, a decrease in the number of barnacles can have a significant effect on the overall fouling community.

Accordingly, processes and compositions are provided for reducing marine fouling. In some embodiments of the presently disclosed subject matter, a process is provided for reducing marine fouling comprising incorporating an anticoagulant other than silicone into a marine coating. In some embodiments, a process is provided for inhibiting the fouling of an object in a marine environment comprising using an anticoagulant other than silicon to inhibit polymerization of barnacle cement such that the ability of the barnacle to adhere to the substrate is lessoned. In some embodiments, a process is provided for inhibiting the fouling of an object in a marine environment comprising, forming on the object, before exposure to the environment, a coating comprising an anticoagulant other than silicon. In some embodiments, a marine foul-release coating composition is provided which comprises an anticoagulant other than silicon.

In some embodiments, the anticoagulant is selected from the group including, but not limited to, glycosaminoglycans (including molecules such as heparin sulfate and dextran sulfate), coumarin-type molecules (including molecules such as DICOUMAROL and WARFARIN), metal chelators (including molecules such as EDTA, EGTA and citrate), plasminogen activators (including molecules such as tissue plasminogen activator) and platelet inhibitors (including molecules such as aspirin).

In some embodiments of the presently disclosed subject matter, a method is provided for identifying compounds useful for reducing marine fouling comprising, measuring either blood coagulation or barnacle cement polymerization in the presence and absence of the compound, wherein a reduction in the blood coagulation or the barnacle cement polymerization in the presence of the compound indicates its usefulness for reducing marine fouling. In some embodiments, the coagulation or the polymerization is measured by measuring a serine protease activity. In some embodiments, the serine protease is a trypsin-like serine protease. In some embodiments, the coagulation or the polymerization is measured by measuring transglutaminase activity.

In some embodiments of the presently disclosed subject matter, a process is provided for reducing marine fouling, comprising incorporating the identified compound that reduces one or both of blood coagulation or barnacle cement polymerization into a marine coating.

In some embodiments of the presently disclosed subject matter, a process is provided for inhibiting the fouling of an object in a marine environment, comprising using the identified compound that reduces one or both of blood coagulation or barnacle cement polymerization to inhibit polymerization of barnacle cement such that the ability of the barnacle to adhere to the object is lessoned.

In some embodiments of the presently disclosed subject matter, a marine foul-release coating composition is provided comprising the identified compound that reduces one or both of blood coagulation or barnacle cement polymerization.

In all embodiments, the presently disclosed anticoagulant compounds and anti barnacle cement polymerization compounds are provided in amounts effective to achieve a reduction in fouling of an object present in a marine environment. A reduction in fouling refers to an amount of that is less than that observed in the absence of an anticoagulant and/or anti-polymerization compound of the presently disclosed subject matter. Determination of effective amounts and/or concentrations of the presently disclosed compounds is well within the skill of one of ordinary skill in the art.

### EXAMPLES

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

### Example 1

### Comparison of Thin, Hard- and Thick, Gummy-Barnacle Plaque

FIG. 1 shows light microscope photographs of the adhesive plaque (base plate with cement layer) of two barnacles (Balanus amphitrite) grown on a VERIDIAN silicone coated glass plate at the Duke University Marine Laboratory. The barnacle on the left (Panel A) expresses the thin, hard phenotype, whereas the barnacle on the right (Panel B) expresses the thick, gummy phenotype. Note the radial canals which can be seen on the thin, hard plaque barnacle but not on the thick, gummy plaque barnacle.

### Example 2

### Images of Fibrin Blood Clots and Barnacle Cement

Blood coagulation and barnacle cement polymerization share several key biochemical characteristics. In both blood coagulation and barnacle cement polymerization, coagulation is achieved through the interaction of proteins (Walker 1971; Davie 1986). In both blood coagulation and barnacle cement polymerization, enzymatic activity is required for coagulation of proteins. Protease activity is critical to blood clotting and barnacle cement polymerization (Dougherty 1996; Dougherty 1997). The activity of serine or serine-like proteases has been shown in both systems (Davie & Rantoff 1964; Dougherty 1996). In both systems the presence of calcium is crucial to enzymatic activity. Disulfide cross-bridges stabilize the enzymes and structural proteins involved in both systems (Davie & Fujikawa 1975; Naldrett 1993). Blood coagulation involves the interaction of fibrin monomers, and barnacle cement polymerization has been previously demonstrated to involve the interaction of at least seven fibrous proteins.

Scanning electron microscopy (SEM) and atomic force microscopy (AFM) of barnacle cement were performed and reveal a fibrous morphology similar to fibrin (see FIGS. 2A-2D). A SEM image of a fibrin blood clot is shown in FIG. 2A. Cross linked fibrin, red blood cells and platelets are shown in the image. Photo credit: Yuri Veklich and John W. Weisel, University of Pennsylvania School of Medicine. An atomic force microscopic image was obtained of residual barnacle cement on a glass slide after barnacle removal from a silicone substrate and subsequent reattachment of the barnacle to a glass slide. After two days in seawater, the barnacle was removed from the glass slide and the residual cement was imaged. The image is shown in FIG. 2B. A confocal light microscopic image of a fibrin blood clot was prepared by adding calcium and thrombin to isolated plasma (Collet et al. 2005). The image is shown in FIG. 2C. An AFM image of a droplet of barnacle cement cured in seawater was obtained as follows. One μl of liquid barnacle cement was obtained and deposited on a glass slide. The droplet was immediately covered by another slide and placed in seawater for 2 days. The image is shown in FIG. 2D.

### Example 3

### Evolutionary Evidence of the Similarity Between Blood Coagulation and Barnacle Cement Polymerization

The process of coagulation is essential to a wide variety of biological phenomena. Coagulation occurs when a material undergoes a transformation from the liquid to the solid phase. Blood coagulation and barnacle cement polymerization are two such biological phenomena. In both of these systems, coagulation serves a key role in survival and reproduction. Coagulation of blood functions to prevent the excessive loss of blood during injury and therefore helps to maintain homeostasis (Davie & Fujikawa 1975). Barnacle cement polymerization allows for a barnacle to permanently adhere to a substrate after metamorphosis to the adult form where it is then able to feed, grow and reproduce (Walker 1971). Ineffective coagulation in both of these systems will lead to death or a failure to reproduce, therefore an effective system of coagulation is essential.

The activity of proteolytic enzymes, such as those involved in blood coagulation, is ubiquitous to biological systems (Neurath & Walsh 1976; Neurath 1986; Krem & Di Cera 2002). The adaptability of these enzymes is shown by their diverse range of functions. The task of proteolytic enzymes ranges from simple digestive function in primitive organisms to complex physiological control in higher organisms (Neurath 1984). Serine proteases, such as those involved in blood coagulation, are found in virtually all organisms from prokaryotes to vertebrates (Kraut 1977). Proteolytic cascades of serine proteases are essential for blood coagulation, the complement cascade and development, among other biological processes. The overall product of the proteolytic cascade is amplification of a small stimulus into a physiological response (Neurath & Walsh 1976; Neurath 1986). This system is efficient and can be regulated.

As selection pressure on effective coagulation is extreme, a system that results in successful coagulation is likely to have evolved early in the history of life, quickly proliferated and been later adapted to fit specific applications such as blood clotting, and barnacle cement polymerization. Similarities between coagulation systems may be due to derivation from a single ancestral clotting mechanism. Alternatively, convergent evolution may have occurred whereby a similar clotting mechanism evolved separately several times, and commonality exists due to similar selective pressures on these systems.

### Example 4

### Comparison of a Fibrin Blood Clot and Polymerized Barnacle Cement by FTIR

FTIR was performed on a fibrin blood clot and polymerized barnacle cement. FIG. 3 shows the FTIR spectra of a fibrin blood clot (top) and of polymerized barnacle cement (bottom). The amide I, II and III region is shown (950-1800 cm⁻¹). Significant peaks are labeled with wavenumbers. The FTIR spectra of polymerized barnacle cement is very similar in both peak position and relative peak intensity to that of clotted fibrin, indicating that the protein configuration and secondary structure of barnacle cement are similar to that of clotted fibrin. The observed similarities in FTIR spectra correlate well with the observed similarity in ultrastructure between barnacle cement and blood clot, as shown by SEM and AFM (see Example 2; FIGS. 2A-2D). The FTIR spectrum of barnacle cement was obtained from residual cement left by whole barnacles on an ATR crystal. The FTIR spectrum of fibrin is from Bramanti et al. 1997.

### Example 5

### Evidence for Trypsin and Fibrinogen in Barnacle Cement

The presence in barnacle cement of the major components of the blood clotting system, trypsin and fibrinogen, was demonstrated using immunostaining. FIGS. 4A-4B shows a Western blotted PVDF membrane immunostained for trypsin. In FIG. 4A, lanes A, B & C are barnacle cement, lane D is a trypsin positive control (4 μg bovine trypsin), and lane E is molecular weight markers (a mix of 10 proteins, 10-225 kDa). Positive staining is observed as dark horizontal bands. Staining in the positive control lane appears primarily at 24 kDa. No staining is observed for molecular weight markers. Positive staining in barnacle cement lanes occur at 90 kDa. In lane C, both the inactive (higher arrow) and activated (lower arrow) forms of the enzyme can be seen. Immunostaining of barnacle cement for trypsin has yielded consistent and reproducible staining at 90 kDa, showing that a trypsin-like molecule is present in barnacle cement. The discrepancy between molecular weight of barnacle cement trypsin versus trypsin positive control, suggests that trypsin in barnacle cement exists as a duplicated form of the molecule.

In FIG. 4B, Western blotted cement proteins (WB) are shown next to SDS-PAGE (reducing conditions) of cement proteins (BC) and molecular weight markers (MW), labeled in kDa. Trypsin immunogen is from bovine pancreas. Immunostaining of barnacle cement for trypsin, a key enzyme responsible for blood coagulation, has yielded consistent and reproducible staining at 90 kDa (see FIG. 4B). The staining at 90 kDa indicates a trypsin-like molecule is present in barnacle cement and that the polymerization of barnacle cement occurs by a similar enzymatic mechanism to that of blood coagulation.

As described herein, trypsin-like serine protease activity is essential to the coagulation of blood (Davie and Rantoff 1964, MacFarlane 1964), and FIGS. 4A-4B show the presence of a trypsin-like serine protease in unpolymerized barnacle cement that is similar to bovine pancreatic trypsin. In addition, the presence of trypsin activity in unpolymerized barnacle cement was verified using BAPNA, an arginine ester substrate useful for specifically detecting trypsin activity. The average trypsin activity measured for unpolymerized barnacle cement was 1.64×10⁻⁶ BAPNA units μl⁻¹ cement (SEM±1.83×10⁻⁷), when incubated for 1 hr at 37° C., pH 8.0 (n=20 barnacles). Therefore, one possible mechanism indicated by the foregoing data for formation of barnacle cement is one in which protease activity activates cement structural precursors, allowing loose assembly with other structural proteins and surface rearrangement.

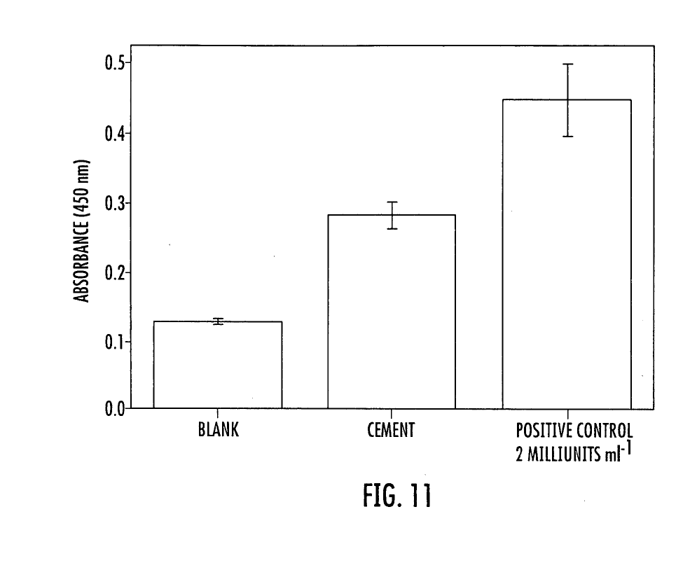

It is generally accepted in the art that the final step in the formation of a stable fibrin blood clot is covalent cross-linking of fibrin monomers by a transglutaminase (factor XIII: Lorand et al. 1962; Lorand et al. 1964). Therefore, the presence of transglutaminase activity was investigated in barnacle cement. Transglutaminase activity in unpolymerized barnacle cement was measured using a commercially available transglutaminase assay kit (see FIG. 11). The results shown in FIG. 11 indicate the presence of transglutaminase activity in unpolymerized barnacle cement. The assay was conducted using a cadaverine coated 96-well plate; n=19 barnacles. The mean and SEM for the blank (no enzyme present) and positive control are shown for comparison. One potential function for the transglutaminase activity in barnacle cement is to covalently cross-link cement monomers. Further, the activity of several of the enzymes involved in blood coagulation are dependent on the presences of calcium. Likewise, polymerization of barnacle cement is inhibited by calcium chelators (such as EGTA and EDTA), indicating the dependence of barnacle cement polymerization on the presence of calcium (see FIG. 6).

FIG. 5 shows an immunostaining experiment for fibrinogen. Fibrinogen is the major structural protein that comprises a vertebrate blood clot. In FIG. 5, positive staining is observed in both dot blots of whole cement droplets (right) and barnacle cement separated using non-denaturing gel electrophoresis and Western Blotted onto a PVDF membrane (left). The quantity of fibrinogen like protein in barnacle cement is roughly 0.5 mg/ml. This estimate is based on a comparison of the staining intensity of cement dot blots to that of controls.

### Example 6

### Anticoagulants Inhibit Polymerization of Barnacle Cement

FIG. 6 shows the protein profile for barnacle cement polymerized in the presence of distilled water (control) or one of 5 anticoagulants (heparin, warfarin, trypsin inhibitor, EGTA or EDTA). Each peak represents an individual protein. For each treatment, 2 μl of either distilled water or anticoagulant was added to 1 μl unpolymerized cement taken from a thick, gummy phenotype barnacle. Polymerization was allowed to proceed for 2 minutes. Samples were analyzed with SDS-PAGE using a 4-20% acrylamide gel. The gel was stained with Coomassie Blue and analyzed using Scion Image.

Barnacle cement polymerization is rapid (less than 2 minutes for gummy barnacles). After polymerization, cement is insoluble and therefore is not observed with SDS-PAGE. For distilled water treatments, very little protein is observed (2 low intensity proteins peaks are shown), which is consistent with polymerization of cement. The number and intensity of proteins is significantly higher in all five anticoagulant treatments, indicating that all five anticoagulants effectively inhibit the polymerization of barnacle cement. The difference between distilled water and anticoagulant treatments is particularly noticeable in the 50-150 kDa region.

### Example 7

### Free Silicon Monomers are Released from a Silicon Foul-Release Coating

FIGS. 7A-7C show GC traces with Mass Spec identification for commercially available silicone foul-release coatings. The traces show that cyclic silicone monomers are being released from the surfaces of the coatings. A) Methanol extraction was performed on VERIDIAN coated glass panels that had been used for barnacle settlement for two years. Each peak on the trace represents a molecule released from the silicone surface. Peaks with matches in the NIST Mass Spec library are labeled with molecular names and structure where available. B) GC Trace with mass spectrometry identification for Dow Corning Silastic T2® silicone, extracted with methanol. Methanol extraction was performed on silicone coated glass panels that had been used as barnacle substrates for two years. The name and structure (if available) of extracted molecules identified in the NIST database are noted. Each peak on the trace represents a molecule released from the silicone surface. The GC/Mass Spec trace provides evidence that silicone monomers are being released from the surface and would therefore be available to interfere with the polymerization of barnacle cement. Accordingly, these data indicate that the silicone monomers act to prevent cement polymerization and cause the thick, gummy cement observed on 30% of barnacles settled on silicone surfaces. C) Mass spec traces for GC peaks shown in (A) and (B) with matching NIST mass spectral library traces.

### Example 8

### Barnacle Reattachment is Decreased in the Presence of Heparin

A barnacle reattachment assay was performed in the presence and absence of heparin. The barnacle reattachment assay (Rittschof et al., under review) allows for rapid assessment of barnacle adhesive strength. In this assay barnacles are grown on non-toxic silicone substrates. Barnacles are removed from the silicone surface using a hand-held mechanical force gauge. Removed barnacles are then placed on another surface to which they are allowed to reattach for one week. As cement production is continuous throughout a barnacle's life, reattachment is possible and strength of adhesion after one week is nearly identical to initial strength of adhesion (when removed from, and reattached to the same silicone substrate). This assay was utilized to determine if a coating on a surface decreases strength of adhesion by removing a barnacle from silicone and allowing it to reattach to test and control surfaces.

Reattachment trials for heparin were conducted by removing a barnacle from a silicone panel and reattaching it to clean glass panels or heparin coated glass panels. Fifteen separate trials were conducted and in nearly every case removal force of reattached barnacles was dramatically and statistically reduced from initial, whereas removal force of controls was statistically similar to initial values. FIG. 8A shows the initial removal force for barnacles removed from T2 (black bars). The reattachment removal force for the barnacles is represented by the gray bars. Barnacles were reattached to glass panels coated with 0 (control), 0.1, 1.0 or 10.0 mg ml⁻¹ heparin for 7 days. The removal force decreased for all barnacles on heparin treatments. A highly significant difference from initial removal force was shown for 10 mg ml⁻¹ heparin (paired samples t-test: p=0.017). A stepwise decrease in the force required for removal was shown as the concentration of heparin was increased from 0.1 to 10 mg ml⁻¹. This pattern was shown on both heparin coated silicone and heparin coated glass.

FIG. 8B shows the mean removal force (±SE) for barnacles reattached to deionized water (dH₂O), sucrose (1 mg ml⁻¹) or heparin (1 mg ml⁻¹) coated T2 silicone over time. The same group of barnacles was successively reattached to the same treatment for 1, 2, 3 and 4 days (in that order); n=10 barnacles for dH₂O and sucrose, n=10 for heparin 1 day, and n=9 for heparin 2, 3 and 4 days. The heparin coated results show a significant difference from the sucrose results for 1 day reattachment (pre-planned sequential Bonferroni pairwise comparison: p<0.05). Statistical comparisons were not conducted on 2, 3, and 4 day trials as there is a potential for the results to be influenced by previous reattachment trials.

FIG. 8C shows the mean removal force (±SE) for barnacles reattached to clean glass (marked as 0.00) and glass coated with heparin at three concentrations. The initial removal force from T2 silicone is shown for comparison. The symbol “*” indicates a significant difference from the deionized water control group (pre-planned sequential Bonferroni pairwise comparison: p<0.05).

FIG. 8D shows data fitted with a sigmoidal dose-response curve to calculate EC₅₀, r²=0.61. Barnacles were reattached for 7 days; n=5 barnacles for each concentration.

### Example 9

### Optical and Atomic Force Microscopy of Barnacle Cement in the Presence of Heparin

Of the anticoagulant compounds tested, heparin showed the most extensive and consistent inhibitory effect on cement polymerization. Barnacle cement is a multicomponent system (Kamino 2006) as is its polymerization (Dickinson 2008). Heparin is a broadly active inhibitor of blood coagulation (Capila and Linhardt 2002). It is generally accepted that the primary mechanism of action of heparin in the blood coagulation cascade is through the binding of antithrombin III, causing accelerated formation of an inactive complex with thrombin and most other coagulation factors (Rosenberg and Damus 1973, Capila and Linhardt 2002). In addition, heparin has the capability to bind directly to thrombin (Pochon et al. 1982, Lambin et al. 1984), and is also known to bind Ca²⁺ (Nieduszynski 1989, Landt et al. 1994, Rabenstein et al. 1995, Karpukhin et al. 2006). Calcium is an essential cofactor for blood coagulation protease and transglutaminase activity.

Accordingly, in the barnacle cement polymerization system of the presently disclosed subject matter, heparin was predicted to have the potential to activate serine protease inhibitors (which are likely to be present in the system to regulate trypsin-like serine protease activity), to bind directly to proteases and cement components and to bind Ca²⁺, thereby reducing the activity of Ca²⁺ dependent enzymes (trypsin-like proteases and transglutaminase). The presently disclosed data and subject matter indicate that trypsin activity in barnacle cement serves a similar biochemical role in cement polymerization as it does in blood coagulation, i.e. activation of structural precursors. Thus, reducing trypsin-like enzyme activity can decrease the number of activated cement precursors and therefore limit the ability of cement proteins to assemble with other structural proteins and for surface rearrangement, resulting in decreased adhesion and altered cement structure (as shown by optical microscopy, for example, see FIGS. 9C-9J).

Specifically, in barnacle reattachment assays heparin decreased removal force in a concentration dependent manner indicating that successive addition of inhibitor can lead to a corresponding decrease in the amount of activated cement precursors that are available for rearrangement with the surface and cross-linking. Optical light microscopy and atomic force microscopy were used to compare the structure of barnacle cement left on clean glass versus barnacle cement left on heparin coated glass. Barnacles were removed from a silicone surface and allowed to reattach to: 1) a clean glass microscope slide, 2) a glass slide coated with 1 mg ml⁻¹ heparin, and 3) a glass slide coated with 10 mg ml⁻¹ heparin. Barnacles were then removed from their reattaching substrate and residual cement was imaged.

FIGS. 9A-9B show AFM images of residual barnacle cement on glass. FIGS. 9C-9J show optical microscope images of residual cement left by barnacles reattaching for one week to: C) clean glass; D) heparin coated glass; E) clean class; F) BSA coated glass (1 mg ml⁻¹); G) BSA coated glass (10 mg ml⁻¹); H) heparin coated glass (1 mg ml⁻¹); and I) & J) heparin coated glass (10 mg ml⁻¹). Residual cement left on clean glass appears as a dense network of interweaving fibers (see, for example, FIGS. 9A & 9C). In contrast, residual cement left on heparin coated glass appeared as longer fibers with minimal networking (see, for example, FIGS. 9B & 9D). Further, time course assays with barnacles reattaching to heparin coated surfaces for 1, 2, 3, and 4 days showed no increase in removal force with increasing time reattached. The results are explainable by either heparin remaining available on the surface for interaction with new cement released by the barnacle, or the adhesion derived from new cement over the course of the 4 day experiment was insufficient to counteract the lack of adhesion in cement initially released by the barnacle upon reattachment and in contact with the heparin.

### Example 10

### Protein Analysis in Barnacle Cement by SDS-Page

Techniques were developed and performed to harvest unpolymerized barnacle cement in microliter quantities. This enabled analysis of the proteins in barnacle cement using SDS-PAGE, which separates proteins based on size. SDS-PAGE provides a protein signature. SDS-PAGE was conducted under reducing conditions on a 4-20% acrylamide gradient gel in the presence and absence of heparin to determine any associated changes in protein signature (See FIG. 10). FIG. 10 shows the SDS-PAGE of unpolymerized barnacle glue, unpolymerized barnacle glue plus heparin, and molecular weight standards. A precast, 4-20% acrylamide gel was used and stained with Coomassie Blue. When heparin is added, the protein signature is significantly different (FIG. 10). Note that the bands smaller than 31 kDa, which correspond to serine proteases and their peptide products, are present for glue only, but do not appear when heparin is added. The number of protein bands in the 85-115 kDa range is increased and of particular note, the 24 kDa protein and all the smaller peptides have disappeared in the presence of heparin.

The analyses show that barnacle cement is composed of at least 12 major proteins, including a protein at 24 kDa and several smaller proteins. These proteins correspond to the molecular weight of the serine proteases found to be active in cement polymerization, and the peptides produced by cleavage of the serine proteases. This result provides evidence that heparin is interfering with the cascade of serine proteases that is active in barnacle cement polymerization.

It will be understood that various details of the presently disclosed subject matter can be changed without departing from the scope of the presently disclosed subject matter. Furthermore, the foregoing description is for the purpose of illustration only, and not for the purpose of limitation.

### REFERENCES

Ansell, J., J. Hirsh, L. Poller, H. Bussey, A. Jacobson, and E. Hylek. 2004. The pharmacology and management of the vitamin K antagonists. Chest 126:204S-233S.
Barnes, H., and J. Blackstock. 1976. Further observations on the biochemical composition of the cement of Lepas fasicularis Ellis & Solander; electrophoretic examination of the protein moieties under various conditions. Journal of Experimental Marine Biology and Ecology 25:263-271
Berglin, M., and P. Gatenholm. 1999. The nature of bioadhesive bonding between barnacles and fouling-release silicone coatings. Journal of Adhesion Science and Technology 13:713-727.
Berglin, M., and P. Gatenholm. 2003. The barnacle adhesive plaque: morphological and chemical differences as a response to substrate properties. Colloids and Surfaces B-Biointerfaces 28:107-117.
Capila, I., and R. J. Linhardt. 2002. Heparin—protein interactions. Angewandte Chemie-International Edition 41:391-412.
Cheung, P. J., G. D. Ruggieri, and R. F. Nigrelli. 1977. A new method for obtaining cement in the liquid state for polymerization studies. Marine Biology 43:157-163.
Davie, E. W. 1986. Introduction to the blood coagulation cascade and the cloning of blood coagulation factors. Journal of Protein Chemistry 5:247-253.
Davie, E. W., and K. Fujikawa. 1975. Basic mechanisms in blood coagulation. Annual Review of Biochemistry 44:799-829.
Davie, E. W., and O. D. Rantoff. 1964. Waterfall sequence for intrinsic blood clotting. Science 145:1310-1312.
Dickinson, G. H. 2008. Barnacle cement: a polymerization model based on evolutionary concepts. Ph.D. Dissertation. Duke University.
Doolittle, R. F. 1984. Fibrinogen and fibrin. Annual Review of Biochemistry 53:195-229.
Dougherty, W. J. 1996. Zinc metalloprotease activity in the cement precursor secretion of the barnacle, Chthamalus fragilis Darwin. Tissue and Cell 28:439-447.
Dougherty, W. J. 1997. Carboxypeptidase activity of the zinc metalloprotease in the cement precursor secretion the barnacle, Chthamalus fragilis Darwin. Comparative Biochemistry and Physiology B-Biochemistry and Molecular Biology 117:565-570.
Hall, M., R. G. Wang, R. van Antwerpen, L. Sottrup-Jensen, and K. Soderhall. 1999. The crayfish plasma clotting protein: A vitellogenin-related protein responsible for clot formation in crustacean blood. Proceedings of the National Academy of Sciences of the United States of America 96:1965-1970.
Holm, E. R., B. Orihuela, C. Kavanagh, and D. Rittschof. 2005. Variation among families for characteristics of the adhesive plaque in the barnacle Balanus amphitrite. Biofouling 21:121-126.
Kamino, K. 2001. Novel barnacle underwater adhesive protein is a charged amino acid-rich protein constituted by a Cys-rich repetitive sequence. Biochemical Journal 356:503-507.
Kamino, K. 2006. Barnacle underwater attachment. Pages 145-165 in A. M. Smith and J. A. Callow, editors. Biological Adhesives. Springer, Berlin.
Kamino, K. 2008. Underwater adhesive of marine organisms as the vital link between biological science and material science. Marine Biotechnology 10:111-121.
Kamino, K., K. Inoue, T. Maruyama, N. Takamatsu, S. Harayama, and Y. Shizuri. 2000. Barnacle cement proteins. Importance of disulfide bonds in their insolubility. Journal of Biological Chemistry 275:27360-27365.
Kamino, K., S. Odo, and T. Maruyama. 1996. Cement proteins of the acorn barnacle, Megabalanus rosa. Biological Bulletin 190:403-409.
Karpukhin, L. E., M. A. Feofanova, L. S. Nikolaeva, M. N. Mamontov, and N. A. Dobrynina. 2006. Complexation of magnesium and calcium ions with heparin. Russian Journal of Inorganic Chemistry 51:908-914.
Kraut, J. 1977. Serine proteases: structure and mechanism of catalysis. Annual Review of Biochemistry 46:331-358.
Krem, M. M., and E. Di Cera. 2002. Evolution of enzyme cascades from embryonic development to blood coagulation. Trends in Biochemical Sciences 27:67-74.
Lambin, P., F. Pochon, and M. Steinbuch. 1984. Heparin and plasma proteinase inhibitors—Influence of heparin on the inhibition of thrombin by alpha-2-macroglobulin. Advances in Experimental Medicine and Biology 167:263-272.
Landt, M., G. L. Hortin, C. H. Smith, A. Mcclellan, and M. G. Scott. 1994. Interference in ionized calcium measurements by heparin salts. Clinical Chemistry 40:565-570.
Lorand, L., A. Jacobsen, and K. Konishi. 1962. Transpeptidation mechanism in blood clotting. Nature 194:1148-1149.
Lorand, L., and K. Konishi. 1964. Activation of fibrin stabilizing factor of plasma by thrombin. Archives of Biochemistry and Biophysics 105:58-67.
MacFarlane, R. G. 1964. An enzyme cascade in the blood clotting mechanism, and its function as a biochemical amplifier. Nature 202:498-499.
Meyer, A., R. Baier, C. D. Wood, J. Stein, K. Truby, E. Holm, J. Montemarano, C. Kavanagh, B. Nedved, C. Smith, G. Swain, and D. Wiebe. 2006. Contact angle anomalies indicate that surface-active eluates from silicone coatings inhibit the adhesive mechanisms of fouling organisms. Biofouling 22:411-423.
Muta, T., and S. Iwanaga. 1996. The role of hemolymph coagulation in innate immunity. Current Opinion in Immunology 8:41-47.
Nakano, M., J. R. Shen, and K. Kamino. 2007. Self-assembling peptide inspired by a barnacle underwater adhesive protein. Biomacromolecules 8:1830-1835.
Naldrett, M. J. 1993. The importance of sulfur cross-links and hydrophobic interactions in the polymerization of barnacle cement. Journal of the Marine Biological Association of the United Kingdom 73:689-702.
Naldrett, M. J., and D. L. Kaplan. 1997. Characterization of barnacle (Balanus eburneus and B. crenatus) adhesive proteins. Marine Biology 127:629-635.
Neurath, H. 1984. Evolution of proteolytic enzymes. Science 224:350-357.
Neurath, H. 1986. The versatility of proteolytic enzymes. Journal of Cellular Biochemistry 32:35-49.
Neurath, H., and K. A. Walsh. 1976. Role of proteolytic enzymes in biological regulation (a review). Proceedings of the National Academy of Sciences of the United States of America 73:3825-3832.
Nieduszynski, I. 1989. General properties of heparin. Pages 51-64 in D. A. Lane and U. Lindahl, editors. Heparin, Chemical and Biological Properties, Clinical Applications. CRC Press, Boca Raton, Fla.
Pochon, F., P. Lambin, and M. Steinbuch. 1982. Heparin and the progressive antithrombin activity of alpha-2-macroglobulin. Thrombosis Research 26:307-316.

Rabenstein, D. L., J. M. Robert, and J. Peng. 1995. Multinuclear magnetic resonance studies of the interaction of inorganic cations with heparin. Carbohydrate Research 278:239-256.
Rice, W. R. 1989. Analyzing tables of statistical tests. Evolution 43:223-225.
Rittschof, D., B. Orihuela, S. Stafslein, J. Daniels, D. Christianson, B. Chisholm, and E. Holm. 2008. Barnacle reattachment: a tool for studying barnacle adhesion. Biofouling 24:1-9.
Rosenberg, R. D., and P. S. Damus. 1973. Purification and mechanism of action of human antithrombin-heparin cofactor. Journal of Biological Chemistry 248:6490-6505.
Sit, P. S., and R. E. Marchant. 2001. Surface-dependent differences in fibrin assembly visualized by atomic force microscopy. Surface Science 491:421-432.
Smith, B. S. 1981. Male characteristics on female mud snails Nassarius obsoletus caused by anti-fouling bottom paints. Journal of Applied Toxicology 1:22-25.
Straw, J., and D. Rittschof. 2004. Responses of mud snails from low and high imposex sites to sex pheromones. Marine Pollution Bulletin 48:1048-1054.
Szczeklik, A., M. Krzanowski, P. Gora, and J. Radwan. 1992. Antiplatelet drugs and generation of thrombin in clotting blood. Blood 80:2006-2011.
Theopold, U., O. Schmidt, K. Soderhall, and M. S. Dushay. 2004. Coagulation in arthropods: defense, wound closure and healing. Trends in Immunology 25:289-294.
Walker, G. 1971. A study of the cement apparatus of the cypris larva of the barnacle Balanus balanoides. Marine Biology 9:205-211.
Walker, G. 1972. The biochemical composition of the cement of the two barnacle species, Balanus hameri and Balanus crenatus. Journal of the Marine Biological Association of the United Kingdom 52:429-435.
Weisel, J. W. 1986. Fibrin assembly—lateral aggregation and the role of the two pairs of fibrinopeptides. Biophysical Journal 50:1079-1093.
Weisel, J. W., Y. Veklich, J. P. Collet, and C. W. Francis. 1999. Structural studies of fibrinolysis by electron and light microscopy. Thrombosis and Haemostasis 82:277-282.
Wiegemann, M., and B. Watermann. 2003. Peculiarities of barnacle adhesive cured on non-stick surfaces. Journal of Adhesion Science and Technology 17:1957-1977.
Woods Hole Oceanographic Institute. 1951. Marine Fouling and its Prevention. 1 edition. United States Naval Institute, Annapolis.
Yan, W., and S. Pan. 1981. The solubilising effect of denaturing chemicals on cement of Balanus reticulatus Utinomi. Oceanologia et limnologia Sinca 12:125-132.

What is claimed is:

1. A process for reducing marine fouling, comprising incorporating an anticoagulant other than silicone into a marine coating.

2. The process of claim 1, wherein the anticoagulant is selected from the group consisting of glycosaminoglycans, coumarin-type molecules, metal chelators, plasminogen activators and platelet inhibitors.

3. The process of claim 2, wherein the anticoagulant is selected from the group consisting of glycosaminoglycans, including heparin sulfate and dextran sulfate.

4. The process of claim 2, wherein the anticoagulant is selected from the group consisting of coumarin-type molecules, including DICOUMAROL and WARFARIN.

5. The process of claim 2, wherein the anticoagulant is selected from the group consisting of metal chelators, including EDTA, EGTA and citrate.

6. The process of claim 2, wherein the anticoagulant is selected from the group consisting of plasminogen activators, including tissue plasminogen activator (tPA).

7. The process of claim 2, wherein the anticoagulant is selected from the group consisting of platelet inhibitors, including aspirin.

8. A process for inhibiting the fouling of an object in a marine environment, which comprises using an anticoagulant other than silicone to inhibit polymerization of barnacle cement such that the ability of the barnacle to adhere to the object is lessoned.

9. The process of claim 8, wherein the anticoagulant is selected from the group consisting of glycosaminoglycans, coumarin-type molecules, metal chelators, plasminogen activators and platelet inhibitors.

10. The process of claim 9, wherein the anticoagulant is selected from the group consisting of glycosaminoglycans, including heparin sulfate and dextran sulfate.

11. The process of claim 9, wherein the anticoagulant is selected from the group consisting of coumarin-type molecules, including DICOUMAROL and WARFARIN.

12. The process of claim 9, wherein the anticoagulant is selected from the group consisting of metal chelators, including EDTA, EGTA and citrate.

13. The process of claim 9, wherein the anticoagulant is selected from the group consisting of plasminogen activators, including tissue plasminogen activator (tPA).

14. The process of claim 9, wherein the anticoagulant is selected from the group consisting of platelet inhibitors, including aspirin.

15. A process for inhibiting the fouling of an object in a marine environment, which comprises forming on the object, before exposure to the environment, a coating comprising an anticoagulant other than silicone.

16. The process of claim 15, wherein the anticoagulant is selected from the group consisting of glycosaminoglycans, coumarin-type molecules, metal chelators, plasminogen activators and platelet inhibitors.

17. The process of claim 16, wherein the anticoagulant is selected from the group consisting of glycosaminoglycans, including heparin sulfate and dextran sulfate.

18. The process of claim 16, wherein the anticoagulant is selected from the group consisting of coumarin-type molecules, including DICOUMAROL and WARFARIN.

19. The process of claim 16, wherein the anticoagulant is selected from the group consisting of metal chelators, including EDTA, EGTA and citrate.

20. The process of claim 16, wherein the anticoagulant is selected from the group consisting of plasminogen activators, including tissue plasminogen activator (tPA).

21. The process of claim 16, wherein the anticoagulant is selected from the group consisting of platelet inhibitors, including aspirin.

22. A marine foul-release coating composition comprising an anticoagulant other than silicone.

23. The coating composition of claim 22, wherein the anticoagulant is selected from the group consisting of glycosaminoglycans, coumarin-type molecules, metal chelators, plasminogen activators and platelet inhibitors.

24. The coating composition of claim 23, wherein the anticoagulant is selected from the group consisting of glycosaminoglycans, including heparin sulfate and dextran sulfate.

25. The coating composition of claim 23, wherein the anticoagulant is selected from the group consisting of coumarin-type molecules, including DICOUMAROL and WARFARIN.

26. The coating composition of claim 23, wherein the anticoagulant is selected from the group consisting of metal chelators, including EDTA, EGTA and citrate.

27. The coating composition of claim 23, wherein the anticoagulant is selected from the group consisting of plasminogen activators, including tissue plasminogen activator (tPA).

28. The coating composition of claim 23, wherein the anticoagulant is selected from the group consisting of platelet inhibitors, including aspirin.

29. A method of identifying compounds useful for reducing marine fouling comprising, measuring either blood coagulation or barnacle cement polymerization in the presence and absence of the compound, wherein a reduction in the blood coagulation or the barnacle cement polymerization in the presence of the compound identifies it as useful for reducing marine fouling.

30. The method of claim 29, wherein the coagulation or the polymerization is measured by measuring a serine protease activity.

31. The method of claim 30, wherein the serine protease is a trypsin-like serine protease.

32. The method of claim 29, wherein the coagulation or the polymerization is measured by measuring transglutaminase activity.

33. A process for reducing marine fouling, comprising incorporating the identified compound of claim 29 into a marine coating.

34. A process for inhibiting the fouling of an object in a marine environment, comprising using the identified compound of claim 29 to inhibit polymerization of barnacle cement such that the ability of the barnacle to adhere to the object is lessoned.

35. A marine foul-release coating composition comprising the identified compound of claim 29.
